(19) United States
(12) Patent Application Publication
(10) Pub. No.: US 2014/0309691 A1
(43) Pub. Date: Oct. 16, 2014

(54) MEDICAL DEVICES CONTAINING SHAPE MEMORY POLYMER COMPOSITIONS

(71) Applicant: Smith & Nephew PLC, London (GB)

(72) Inventors: Malcolm Brown, Otley (GB); Horacio Montes de Oca Balederas, York (GB); Michael Andrew Hall, Heslington (GB); Alan William Bull, York (GB); David Franklin Farrar, Heslington (GB); Philip Caton-Rose, Bradford (GB); Philip David Coates, Bradford (GB); Glen Thompson, Bradford (GB); Michael Martyn, Bradford (GB); Ian MacMillian Ward, Leeds (GB); Mark James Bonner, Leeds (GB); Peter John Hine, Leeds (GB)

(21) Appl. No.: 14/350,030

(22) PCT Filed: Oct. 5, 2012
(86) PCT No.: PCT/GB2012/052470

§ 371 Date: Apr. 4, 2014

(30) Foreign Application Priority Data
Oct. 5, 2011 (GB) 1117214.5
Oct. 5, 2011 (GB) 1117216.0
Oct. 5, 2011 (GB) 1117217.8
Oct. 5, 2011 (GB) 1117218.6
Oct. 5, 2011 (GB) 1117219.4
Oct. 5, 2011 (GB) 1117220.2
Oct. 5, 2011 (GB) 1117222.8
Oct. 5, 2011 (GB) 1117223.6
Oct. 5, 2011 (GB) 1117224.4
May 29, 2012 (GB) 1209510.5

(51) Int. Cl. A61B 17/04

(52) U.S. Cl. CPC A61B 17/0401; A61B 2017/042
USPC 606/232

(57) ABSTRACT

The present invention relates at least in part to surgical devices which comprise a shape memory polymer material composition. Particularly, although not exclusively, the present invention relates to a fixation device e.g. an anchor device e.g. a suture anchor which comprises a shape memory material. Included in the present invention are anchor devices e.g. suture anchors which are formed entirely of a shape memory polymer material. Embodiments of the present invention comprise hybrid suture anchors, particularly suture anchors which are formed from a shape memory polymer material and a non-shape memory material. Methods of securing an anchor in a bone or tissue are also included in the present invention.

### FIELD OF THE INVENTION

The present invention relates at least in part to surgical devices which comprise a shape memory polymer material composition. Particularly, although not exclusively, the present invention relates to a fixation device e.g. an anchor device e.g. a suture anchor which comprises a shape memory material. Included in the present invention are anchor devices e.g. suture anchors which are formed entirely of a shape memory polymer material. Embodiments of the present invention comprise hybrid suture anchors, particularly suture anchors which are formed from a shape memory polymer material and a non-shape memory material. Methods of securing an anchor in a bone or tissue are also included in the present invention.

### BACKGROUND TO THE INVENTION

Suture anchors and sutures are used in a number of orthopaedic procedures to reattach soft tissue to bone. Examples of procedures that involve the use of anchors and/or sutures include: procedures in the shoulder for example rotator cuff repair and treatment of glenohumeral instability (e.g. repair of Bankart and SLAP lesions); procedures in the hip region e.g. repair of the labrum in the hip and procedures in the foot and ankle region e.g. repair of ligaments/tendons.

Suture anchors usually fail because the anchor pulls out, the suture cuts out the eyelet of the anchor or simply the suture breaks.

Often, it is desirable to use suture anchors with the smallest possible diameter, yet which still provide adequate fixation strength, particularly when carrying out repairs on joints with limited bone volume. Smaller anchors require smaller drill holes, and are less traumatic for the patient. They also provide more flexibility to the surgeon in positioning the anchor or anchors. A problem associated with reducing the size of an anchor is that there is generally a reduction in fixation strength. This reduction in fixation strength generally limits the minimum size of anchors that can be used. This problem can be worsened if the quality of the bone is poor, which may especially be the case in older patients. A further disadvantage of current methods and systems is caused by the accidental drilling of oversized holes. This can occur if the drill is inadvertently moved or allowed to “wobble” during drilling. If a conventional anchor is then placed in an oversized hole the fixation strength can be greatly reduced.

Conventional suture anchors are typically formed from metals, bioresorbable polymers (such as polylactide or polylactide-co-glycolide) (PLGA) or non-bioresorbable polymers (such as PEEK). To improve fixation in bone the anchor design may include external ridges, ribs, fins or barbs; alternatively it may include an external screw thread. Other devices may use a pin to mechanically expand flanges on the anchor that aid fixation.

Due to the complex geometry of the anchors they are usually manufactured by injection moulding techniques, hence only a limited amount of molecular orientation can be imparted to the polymeric implant.

There remains a need to provide suture anchors and other fixation devices which can function in a range of bone qualities. There remains a further need to provide suture anchors and other fixation devices which are smaller in diameter than existing anchors and which offer equal or better fixation strength.

It has been proposed that shape memory polymers (SMPs) can be used in tissue anchors to improve fixation. International Patent with publication number WO 2008/118782 (Cotton et al,) describes an anchor made from polylactide-co-glycolide (PLGA) and calcium carbonate where the device deforms at body temperature to increase fixation.

U.S. Pat. No. 8,069,858 (Gall, Medshape Solutions, Inc) describes an anchor that comprises a shape memory polymer portion that is triggered by a physical force below the activation temperature of the polymer. The device described in U.S. Pat. No. 8,069,858 appears to require mechanical activation in order for the device to change shape.

It is an aim of embodiments of the present invention to address the disadvantages of the prior art.

### SUMMARY OF THE INVENTION

In a first aspect of the present invention, there is provided a fixation device for use to secure itself and/or a further device in a cavity, the fixation device comprising a Shape Memory Polymer (SMP) material, wherein the SMP material is capable of radial expansion when activated such that the fixation device expands radially in at least a section of its length.

Aptly, the fixation device is selected from a pin, a tac, a screw, a rod, a nail, a plate, an anchor and a wedge.

Aptly, the fixation device is a surgical device.

Aptly, the fixation device is a suture anchor.

Aptly, the fixation device is capable of undergoing radial expansion and longitudinal contraction and/or a geometry change when the SMP material is activated. Aptly, the fixation device undergoes a geometry change upon activation. Aptly, the fixation device undergoes a dimensional change upon activation.

In one embodiment, the suture anchor comprises an anchor body comprising a distal portion and a proximal portion. Aptly, the anchor body comprises a passage extending from the distal portion toward the proximal portion. Aptly, the passage is a through passage.

Aptly, the anchor body comprises one or more circumferential ribs. In one embodiment, the circumferential ribs extend from the outward surface of the anchor body following activation of the SMP material. Aptly, the circumferential ribs only protrude from an outer surface of the device upon activation of the SMP material.

Aptly, the fixation device comprises screw threads along its length.

Aptly, the fixation device is formed integrally from a single piece of SMP material.

Aptly, the fixation device comprises a portion comprising the SMP material and a further portion comprising a non-SMP material. Aptly, the further portion consists of the non-SMP material. In one embodiment, the further portion is formed by a process of overmoulding. In one embodiment, the further portion is formed by injection moulding.

As used herein, the term “non-SMP material” is taken to include materials which do not possess shape memory qualities, i.e. do not change shape back towards an initial shape when heated or otherwise activated. Examples of such materials as described herein. Aptly, the non-SMP material may be a polymer which has not undergone programming to impart shape memory qualities thereto. Aptly, the non-SMP material comprises a plastic e.g. a moulded plastic.

Aptly, the fixation device comprises one or more circumferential ribs composed of the non-SMP material.

Aptly, the fixation device is for the delivery of a fluid. In one embodiment, the device comprises a chamber which comprises a fluid. Aptly, the radial expansion is capable of causing the fluid to be released from the chamber to the environment surrounding the device.

Aptly, the device comprises an inner portion which comprises the SMP material.

In an embodiment, the device comprises one or more limbs which extend outwardly upon activation of the SMP material. Aptly, the limbs comprise the SMP material.

Aptly, the SMP material comprises a polymer selected from the group consisting of polymethyl methacrylate (PMMA), polyethyl methacrylate (PEMA), polyacrylate, poly-alpha-hydroxy acids, polycapropactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, polyurethanes, and copolymers or polymer blends thereof.

Aptly, the SMP material comprises a polyester.

Aptly, the SMP material comprises a polylactide. Aptly, the SMP material comprises poly(L-lactide) e.g. a co-polymer thereof. Aptly, the SMP material comprises a poly(D,L-lactide) co polymer. In one embodiment, the SMP material comprises a poly(DL-lactide-co-glycolide) (PDLGA) co polymer e.g. a PDLGA co polymer having a ratio of 85 (DL-lactide):15 (glycolide). Alternatively, the ratio is e.g. 70:30, 75:25, 80:20 or 90:10.

In one embodiment, the SMP material further comprises a filler. Aptly, the SMP material comprises a bioceramic material. Aptly, the bioceramic is selected from a calcium phosphate, a calcium carbonate and a calcium sulphate and combinations thereof. Aptly, the SMP material is buffered to enhance strength retention. Suitable buffering agents include calcium carbonate.

Aptly, the SMP material further comprises a plasticiser, a bioactive agent and/or a pharmaceutical agent. Further details of suitable plasticisers, bioactive agents and pharmaceutical agents are disclosed herein. Aptly, the non-SMP material comprises a biocompatible polymer and/or a biocompatible composite. In one embodiment, the non-SMP material is resorbable.

In one embodiment, the non-SMP material is selected from polylactide, polyglycolide, polycaprolactone, poly(lactide-co-glycolide), polydioxanone, polyurethane, a blend of one or more thereof, and a copolymer thereof. Aptly, the non-SMP material is a polymer that has not undergone programming to impart shape memory properties.

Aptly, the non-SMP material is non-resorbable. In one embodiment, the non-SMP material is a non-resorbable polymer selected from the group consisting of polyetheretherketone (PEEK), a polyurethane and a polyacrylate.

Aptly, the device has a diameter of less than about 3 mm. In one embodiment, the device has a diameter of approximately 2 mm or less e.g. 1 mm, 1.2 m, 1.5 mm or 1.7 mm.

In a further aspect of the present invention, there is provided a method of repairing a soft tissue comprising; placing a device as described herein and having a flexible member coupled thereto in a cavity in a bone, passing the flexible member through a soft tissue located adjacent to the bone and tying the flexible member to secure the soft tissue to the bone; and activating the SMP material such that the device undergoes a radial expansion in at least a section of its length.

Aptly, the method is carried out on a human patient. Aptly, the method is carried out on an animal patient.

Aptly, the step of activating the SMP material comprises applying heat to the SMP material. Aptly, the method comprises contacting the SMP material with a heated probe.

Aptly, the method comprises a first step of forming the cavity in the bone and placing the device in the cavity. Aptly, the flexible member is a suture.

Aptly, the soft tissue is selected from a tendon, a ligament, a muscle, and cartilage and a combination thereof. Aptly, the method is for the repair of a rotator cuff.

In one embodiment, the method is for the repair of an anterior cruciate ligament (ACL). Aptly, the method is for the treatment of glenohumeral instability e.g. repair of Bankart and SLAP lesions. Aptly, the method is for the treatment of hip labral tear.

According to an aspect of the present invention, there is provided shape memory sutures that expand in thickness and shrink in length suitable for use in wound closure. Aptly, the suture is mechanically attached to a fixation device described herein. Aptly the suture is inserted with the fixation device e.g. anchor into a cavity drilled on the bone and passed through the tissue to be fixed.

In an embodiment, multiple anchors are provided to fixate multiple sutures.

### BRIEF DESCRIPTION OF DRAWINGS

Embodiments of the present invention will now be described hereinafter, by way of example only, with reference to the accompanying drawings in which:

FIG. 1 illustrates an embodiment of the present invention situated in Sawbones;

FIG. 2 illustrates the push out force of a shape memory polymer material (SMP) suture anchor as illustrated in FIG. 1 after 9 days of immersion. A poly (DL-lactide-co-glycolide) (85:15) (PLC) die drawn rod 9 mm in diameter was inserted into a hole drilled onto Sawbones (20pcf), ensuring that it remained “loose” i.e a force of ON was initially required to pull the rod out of the hole. The PLC rod comprises 35% w/w calcium carbonate. The Sawbones with the PLC rod (anchor) was immersed in water at 37 C for 9 days. The push-out force was measured with the Instron apparatus operated at 1 mm/min;

FIGS. 3a and 3b illustrate an SMP suture anchor of the present invention which shortens and expands radially upon activation to fixate into the surrounding bone;

FIGS. 4a to 4d illustrate alternative embodiments of a suture anchor according to the present invention comprising multiple fixation ribs;

FIGS. 5a to 5c illustrate alternative embodiments of a suture anchor according to the present invention comprising upward directing fixation ribs;

FIGS. 6a to 6b illustrate alternative embodiments of a suture anchor according to the present invention comprising SMP levering elements;

FIGS. 7a and 7b illustrate an SMP suture;

FIGS. 8a and 8b illustrate an SMP anchor comprising fixation elements;

FIGS. 9a and 9b are cross sectional views of the anchor of FIGS. 8a and 8b;

FIGS. 10a and 10b illustrate an SMP anchor with multiple axle fixation elements;

FIGS. 11a and 11b are cross sectional views of FIGS. 10a and 10b;

FIGS. 12a and 12b show an SMP anchor with fixation elements;

FIGS. 13a and 13b are cross sectional views of FIG. 12c-12b;

FIGS. 14a and 14b show an SMP anchor with folded fixation elements;

FIGS. 15a and 15b show an SMP anchor with fixation elements contained within an oriented section of the device;

FIGS. 16a and 16b show an SMP clip;

FIGS. 17a to 17d show various tissue closure SMP devices according to the present invention;

FIG. 17e illustrates performance data from SMP sutures;

FIGS. 18a and 18b illustrate an embodiment of the present invention which comprises a SMP fluid delivery device;

FIGS. 19a and 19b illustrate an embodiment of the present invention which comprises a SMP fluid delivery device;

FIGS. 20a and 20b are cross sectional views of FIGS. 19a and 19b;

FIG. 21 illustrates a SMP suture sleeve which forms a fixation aid post insertion;

FIG. 22 illustrates an SMP anchor with a suture hole and activation region (hole);

FIGS. 23a to 23c illustrate an embodiment of the present invention comprising a SMP suture anchor which is capable of relaxing in the longitudinal direction following implantation;

FIGS. 24a to 24c illustrate an embodiment of the present invention comprising an anchor with a dedicated SMP portion which directs a portion of the anchor into a fixation position;

FIG. 25 illustrates an embodiment of the present invention comprising an SMP anchor with a dedicated SMP portion which clamps and fixes the suture following relaxation of the polymer;

FIGS. 26a and 26b illustrate an embodiment of the present invention comprising an SMP suture containing two different areas of memory orientation. Following relaxation a portion of the SMP relaxes in the longitudinal direction to fix the anchor in place;

FIG. 27a illustrates an embodiment of the present invention comprising an SMP anchor with multiple suture eyelets in a vertical direction;

FIG. 27b illustrates an embodiment of the present invention comprising an SMP anchor with suture eyelets in a longitudinal direction;

FIG. 27c illustrates an embodiment of the present invention comprising an SMP anchor with shaped grooves to accommodate a suture material;

FIGS. 28a and 28b illustrate an embodiment of the present invention comprising an anchor with an SMP pin which directs a portion of the anchor into a fixation position following relaxation;

FIGS. 29a and 29b illustrate an embodiment of the present invention comprising an SMP suture tack with a SMP portion which shortens in the vertical direction and lengthens in the longitudinal direction to fixate the device;

FIG. 30a illustrates an embodiment of the present invention comprising an anchor with an SMP collar which fixates the suture following relaxation;

FIGS. 30b and 30c illustrate an embodiment of the present invention comprising a barbed suture anchor with an SMP portion running longitudinally through the length of the device. Following relaxation of the SMP the barbed portion is forced outwards causing fixation;

FIG. 30c illustrates an alternative embodiment of FIG. 30b;

FIGS. 31a and 31b illustrate an embodiment of the present invention comprising an injection moulded pronged suture anchor. Shape memory properties are added to the prongs via compression moulding;

FIGS. 32a and 32b illustrate an embodiment of the present invention comprising a suture anchor with an SMP portion which causes the suture to be secured by a fixation element following relaxation of the SMP;

FIG. 32c illustrates a SMP tube used in FIG. 10a-10b;

FIGS. 33a and 33b illustrate an embodiment of the present invention comprising a suture anchor with an SMP element which upon relaxation causes the device to fixate (FIG. 33a-post fixation);

FIGS. 34a and 34b illustrate an embodiment of the present invention comprising a suture anchor with an SMP portion. Following relaxation in the longitudinal direction the anchor fixates into the tissue;

FIGS. 34c and 34d illustrate an embodiment of the present invention comprising a suture anchor with an internal SMP feature which causes the device to fixate following relaxation;

FIG. 35 illustrates the device of three embodiments of the present invention which comprise an SMP portion;

FIG. 36 is a graphical representation of a slotted SMP rod prototype according to the present invention;

FIG. 37 is a graph showing the pull-out test results in 10PCF Sawbones, 2.6 mm holes as described in Example 7;

FIG. 38 is a graph showing the pull-out testing of SMP rod anchors in 10PCF Sawbones in standard and oversized holes as described in Example 8;

FIG. 39 is a graph showing the results of pull-out testing in laminated 15/30PCF “Sawbones” foam as described in Example 9;

FIG. 40 is a graphical representation showing pre- and post-recovery appearance of tested devices—Left hand side: Hybrid Anchor Prototype 3; right hand side 2.7 mm SMP rod anchor;

FIG. 41 illustrates a knotless suture anchor produced using methods described in Example 12;

FIG. 42 illustrates a suture anchor as described in Example 13;

FIG. 43 illustrates a suture as described in Example 14;

FIG. 44 illustrates an embodiment of the present invention which comprises an SMP portion and a non-SMP portion;

FIG. 45 shows further views of the non-SMP portion of the device of FIG. 44;

FIG. 46 illustrates a further embodiment which comprises a suture anchor device which is composed entirely of an SMP material;

FIG. 47 illustrates a tool for aiding insertion of the suture anchor illustrated in FIG. 46. The tool also includes a heater; and

FIG. 48 shows further views of the suture anchor illustrated in FIG. 46.

### DETAILED DESCRIPTION OF EMBODIMENTS OF THE INVENTION

Further details of embodiments of the present invention are described below.

The present invention comprises the use of a shape memory polymer (SMP) material. In an embodiment, the SMP material resides in a deformed state below a certain temperature, known as the glass transition temperature (Tg) and is activatable from the deformed state to the relaxed state above this temperature. Generally, polymeric materials that display shape memory properties show a large change in modulus of elasticity at the glass transition temperature (Tg). Shape-memory properties are utilized by taking advantage of this characteristic. Namely, a macroscopic body of polymeric shape memory material to which a definite shape (the original shape) has been imparted by a common method for moulding plastics, can be softened by providing the article with energy and heating to a final temperature (Tf) higher than the Tg of the polymer, but lower than the melting temperature (Tm). At this temperature, the material can be deformed so as to form a different macroscopic shape (the deformed state). In the deformed state an oriented polymer network is formed. The polymeric material is then cooled to a temperature lower than the Tg, whilst maintaining its deformed state.

A device of the invention comprises a polymeric shape memory material. Shape memory polymers, which can be resorbable or non-resorbable, are known in the art and any biocompatible polymeric shape memory material can be used in the context of the present invention. Aptly, the SMP material comprises a polymer selected from the group consisting of polymethyl methacrylate (PMMA), polyethyl methacrylate (PEMA), polyacrylate, poly-alpha-hydroxy acids, polycaprolactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, polyurethanes, and copolymers or polymer blends thereof.

Aptly, the SMP material comprises a polylactide. In one embodiment, the SMP material comprises poly(L-lactide). In one embodiment, the SMP material comprises poly(D-lactide).

In one embodiment, the SMP material comprises a poly(D,L-lactide) co polymer.

Aptly, the SMP material comprises a poly(DL-lactide-co-glycolide) (PDLGA). Aptly, the SMP material comprises polyglycolide.

Aptly, the SMP material comprises polycaprolactone and/or a co-polymer comprising polycaprolactone.

Aptly, the SMP material comprises an L-lactide/DL-lactide co-polymer.

Aptly, the SMP material comprises lactide/caprolactone copolymer.

Aptly, the SMP material comprises a poly(L-lactide) and polyglycolide copolymer.

In the context of the present invention, deformation of the polymeric shape memory material is generally achieved prior to implantation of the device, generally during manufacture. The input of heat sufficient to reach Tf achieved using electrical and/or thermal energy sources and this is followed by deformation of the polymeric material. Deformation leads to an oriented polymer network and can be achieved by processes including zone drawing, hydrostatic extrusion, die drawing, compression flow molding, thermoforming, rolling and roll drawing.

When the polymeric material is heated again to a temperature higher than the glass transition temperature of the SMP material, but lower than the Tm, the deformed state disappears and the polymeric material relaxes to recovered its original shape. The input of energy necessary to cause the polymeric material to relax from its deformation state to its relaxed state is known as activation. The glass transition temperature of the polymer material will vary based on a variety of factors, such as molecular weight, composition, structure of the polymer, and other factors known to one of ordinary skill in the art and may be in the region of between 35-60° C. or greater. Aptly, the glass transition temperature is up to about 130° C. Aptly, the glass transition temperature is about 70° C. or more e.g. 80° C., 90° C., 100° C., 110° C. or 120° C.

Embodiments of the present invention relate to devices which alter shape in situ through recovery of a SMP material towards its original shape. As used herein the terms “recovery” and “recover” are interchangeable with the terms “relaxation” and “relax” and are terms well known to the person skilled in the art.

In one embodiment, the fixation device is selected from a pin, a rod, a nail, a screw, a plate, an anchor and a wedge.

Aptly, the fixation device is an intramedullary nail.

In one embodiment, the fixation device is an anchor. Aptly, the anchor is a suture anchor. Aptly, the anchor is a knotless suture anchor. Aptly, the anchor comprises one or more grooves on an outer surface thereof which are sized to accommodate one or more sutures therein. Aptly the suture anchor comprises a non-SMP material component and an SMP material component. Aptly, the non-SMP material component comprises the grooves. In one embodiment, the grooves extend the length of the device on two opposing outer surfaces of the anchor and across a distal end of the anchor.

In one embodiment, the suture anchor is composed entirely of an SMP material and the SMP material component comprises the grooves.

Embodiments of the present invention may provide an advantage over prior art suture anchors in that the anchor eyelet failure may be reduced and/or the failure load increased. Embodiments of the present invention may provide a suture anchor which has a greater fixation strength in poor-quality and/or low density bone e.g. osteoporotic bone.

Tissue anchors such as suture anchors may fail during insertion when they are screwed in. Embodiments of the present invention may also provide an advantage in that, since they do not require screwing in during insertion, failure may be avoided.

Compared with standard conventional anchors, embodiments of the present invention provide an anchor which has a smaller cross-section and/or length while maintaining equivalent or higher pull-out strength.

In an embodiment, the present invention provides a fixation device e.g. a suture anchor which comprises a portion comprising an SMP material and a portion comprising a non-SMP material. Such an embodiment may provide an advantage that initial fixation may be achieved by the non-SMP material portion and subsequently the fixation strength of the device may be further enhanced over time by the SMP material portion. In one embodiment, the SMP material is activated at body temperature (e.g. approximately 37° C.) and the SMP material portion expands when placed in the body to further fix the device in place.

Fixation devices comprising an SMP material portion and a non-SMP material portion may also be advantageous in that complex design features and shapes can be formed by conventional injection moulding of a non-SMP material and fixation can be enhanced by the SMP material component which can be a simple shape, for example a rod or cylinder, made by a process such as die-drawing. Aptly, the device can be made by placing an SMP material component in a mould and injection moulding a non-SMP material into the mould, thus overmoulding the non-SMP material onto the SMP material component.

The non-SMP elements of the device may be made of any biocompatible polymer or composite, either resorbable or non-resorbable. Examples of resorbable materials include polylactide, polyglycolide, polycaprolactone, poly(lactide-co-glycolide), polydioxanone, polyurethane or any blend or copolymer of these materials. Examples of non-resorbable polymers include polyetheretherketone (PEEK), polyurethanes, polyacrylates etc. the polymer may be blended with fillers including bioceramics such as, for example, calcium phosphates, calcium carbonates, calcium sulphates and the like.

The SMP components may be made of any of the polymers described herein suitably processed to impart shape memory properties. Methods of imparting shape memory properties include processes to orient the polymer chains and include die drawing, zone drawing, hydrostatic extrusion, rolling, roll drawing, compression moulding. The SMP component may also include plasticizers to modify the glass transition temperature/activation temperature. It may also include other additives such as iron oxide nanoparticles to enable activation by a magnetic field. Activation of the SMP component may be by heat (including body temperature), absorption of a plasticizer such as water, electromagnetic field, ultrasound or any other method or combination of methods.

Compared with standard conventional anchors the SMP anchor allows the use of a smaller drill hole/anchor while maintaining equivalent or higher pull-out strength. Another advantage is greater fixation strength in poor-quality or low density bone (e.g. osteoporotic bone). Yet another advantage is that fixation can be achieved in oversized holes—for example if the hole is accidentally over-drilled. The device can be very simple and in some embodiments does not require feature such as ribs, ridges or barbs. Aptly, the device is easier to insert than conventional fixation devices. In other embodiments, the device may comprise one or more barbs, ribs or ridges. Aptly, the barbs, ribs or ridges are used to improve the fixation of the device once the SMP material has been activated.

An advantage of embodiments of the present invention which comprise SMP material components and non-SMP components is that initial fixation can be achieved by a conventional non-SMP part and then fixation strength can be further enhanced over time by an SMP component that is activated at body temperature. This does not necessarily require any external heating/energy source to activate the SMP.

Another advantage is that complex design features and shapes can be made by conventional injection moulding of a non-SMP and fixation further enhanced by the SMP component that can be a very simple shape such as a rod or cylinder made by processes such as die-drawing. No complex process or apparatus is required to programme the SMP device.

The devices of the present invention can be manufactured using known techniques for forming SMP materials for example die drawing.

Aptly, the device may be manufactured using a method which includes a step of overmoulding non-SMP material and therefore producing a device which includes non-SMP material portions.

Aptly, the device may be manufactured by a method comprising cold forging so as to impart a complex shape to the device.

Details of process for the manufacture of hybrid devices can be found in our co-pending patent applications which have a common priority to the present patent application. The subject matter of our co-pending patent applications and the priority applications are hereby incorporated herein by reference in their entirety.

In an embodiment, one or more active agent is incorporated into the device. Suitable active agents include bone morphogenic proteins, antibiotics, anti-inflammatories, angiogenic factors, osteogenic factors, monobutyrin, omental extracts, thrombin, modified proteins, platelet rich plasma/solution, platelet poor plasma/solution, bone marrow aspirate, and any cells sourced from flora or fauna, such as living cells, preserved cells, dormant cells, and dead cells. It will be appreciated that other bioactive agents known to one of ordinary skill in the art may also be used. Aptly, the active agent is incorporated into the polymeric shape memory material, to be released during the relaxation or degradation of the polymer material. Advantageously, the incorporation of an active agent can act to combat infection at the site of implantation and/or to promote new tissue growth.

Aptly, the SMP material comprises a filler. In one embodiment, the filler comprises an inorganic component. Aptly, the filler comprises calcium carbonate, calcium hydrogen carbonate, calcium phosphate, dicalcium phosphate, tricalcium phosphate, magnesium carbonate, sodium carbonate, hydroxyapatite, bone, phosphate glass, silicate glass, sodium phosphate, magnesium phosphate, barium carbonate, barium sulphate, zirconium carbonate, zirconium sulphate, zirconium dioxide, bismuth trioxide, bismuth oxychloride, bismuth carbonate, tungsten oxide and combinations thereof.

Aptly, the SMP material comprises approximately 0.5% or greater by weight of a filler as described herein. Aptly, the SMP material comprises 0.5%, 1%, 2%, 3%, 5%, 10%, 15%, 20%, 25%, 30%, 35%, 40% or greater by weight of a filler.

The present invention contemplates the use of electrical and thermal energy sources to heat the polymeric material. However, the polymer material could be relaxed via other methods known to those of ordinary skill in the art, including, but not limited to the use of force, or mechanical energy, and/or a solvent. Any suitable force that can be applied either preoperatively or intra-operatively can be used.

One example includes the use of ultra sonic devices, which can relax the polymer material with minimal heat generation. Solvents that could be used include organic-based solvents and aqueous-based solvents, including body fluids. Care should be taken that the selected solvent is not contra indicated for the patient, particularly when the solvent is used intra-operatively. The choice of solvents will also be selected based upon the material to be relaxed. Examples of solvents that can be used to relax the polymer material include alcohols, glycols, glycol ethers, oils, fatty acids, acetates, acetylenes, ketones, aromatic hydrocarbon solvents, and chlorinated solvents.

Aptly, the SMP material portion of the device is activated by way of heating when inserted into the cavity. In one embodiment, the SMP portion of the device is activated by contacting the SMP material portion with a heated probe or the like.

In one embodiment, the SMP material portion is activated by contact with an aqueous media which has a temperature of about 37° C. i.e. body temperature. Aptly, the SMP material comprises a plasticizer which lowers the Tg of the SMP material so that it is activated by contact with an aqueous media having a temperature close to body temperature e.g. about 37° C. Thus, in one embodiment the SMP material portion is capable of activation upon insertion into a patient's body.

Reduction of the SMP material's Tg may be achieved by inclusion of a plasticiser. Aptly, the SMP material comprises a plasticiser. Plasticisers or mixtures thereof suitable for use in the present invention may be selected from a variety of materials including for example organic plasticisers and those that do not contain organic compounds.

Aptly, the plasticiser is selected from DL-lactide, L-lactide, glycolide, ε-Caprolactone, N-methyl-2-pyrolidinone and a hydrophilic polyol e.g. poly(ethylene) glycol (PEG).

Plasticisers or mixtures thereof suitable for use in the present invention may be selected from a variety of materials including organic plasticisers and those that do not contain organic compounds.

Aptly, the plasticiser is an organic plasticiser e.g. a phthalate derivatives such as dimethyl, diethyl and dibutyl phthalate; a polyethylene glycol with a molecular weight e.g. from about 200 to 6,000, glycerol, glycols e.g. polypropylene, propylene, polyethylene and ethylene glycol; citrate esters e.g. tributyl, triethyl, triacetyl, acetyl triethyl, and acetyl tributyl citrates, surfactants e.g. sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof.

Aptly, the plasticiser is selected from a citrate ester; a polyethylene glycol and dioxane.

In an embodiment, the device comprises reinforced polymeric material. Aptly, the reinforced polymeric material comprises a composite or matrix including reinforcing material or phases e.g. fibers, rods, platelets and fillers. Aptly, the polymeric material can include glass fibers, carbon fibers, polymeric fibers, ceramic fibers and/or ceramic particulates. Other reinforcing material or phases known to one of ordinary skill in the art could also be used.

Once the device has been produced, it may be sterilized for example by exposing it to radiation (e.g. gamma radiation) or treating it with gases (e.g. chemical sterilization such as exposure to ethylene oxide gas). Methods of sterilizing devices are known in the art, and the skilled person may select a method appropriate for the device in question.

### DESCRIPTION OF EMBODIMENTS

In the drawings like reference numerals refer to like parts.

Amorphous poly(D,L lactide-co-glycolide) with 35% w/w CaCO₃ (PLC) fibres were prepared using a twin screw extruder. The fibres were pelletised and consolidated into isotropic long cylindrical rods with various diameters ranging from 5 mm to 20 mm using a ram extrusion technique. Oriented rods 3 mm and 9 mm in diameter were prepared by die drawing the isotropic rods (5 mm and 20 mm, respectively) using a conical die at 60 C and a drawing speed of 20 mm/min.

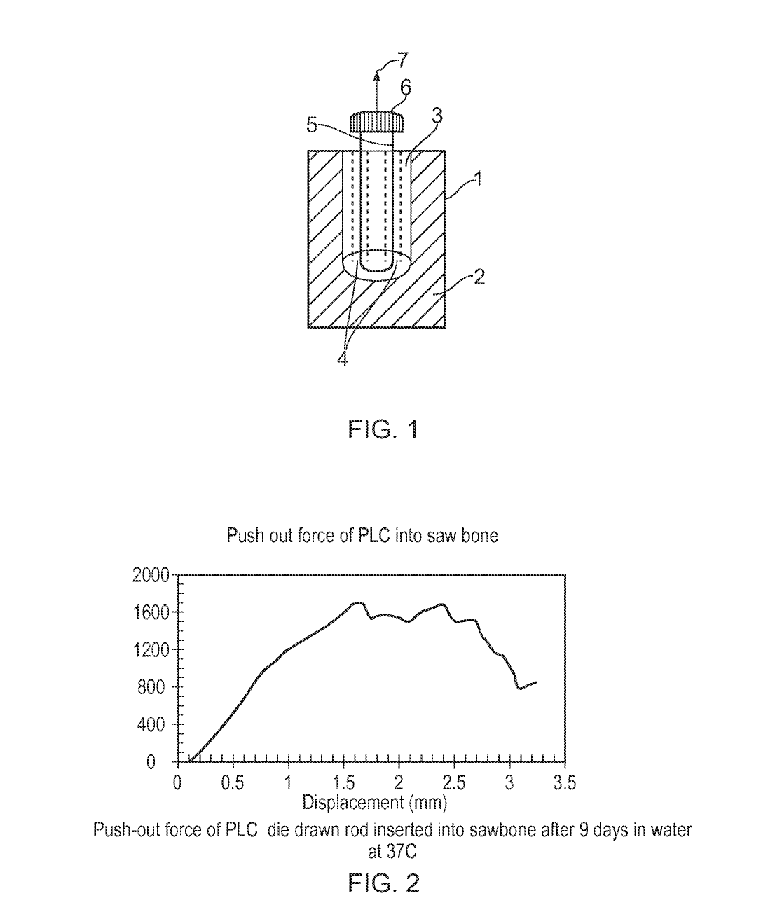

FIG. 1 illustrates an artificial construct 1 which was made to replicate the in-vivo use of the proposed suture anchors. Two holes 4 were drilled along the long axis of a die drawn cylindrical PLC rod 9 mm in a diameter 3. A polyester suture 5 was inserted (making a U turn) through both holes mimicing a suture anchor.

The rod was inserted into a hole drilled onto Sawbones™ (20pcf) 2 ensuring that it remained “loose” i.e a force of ON was initially required to pull the rod out of the hole. The Sawbones™ with the rod in the hole was immersed into water at 80° C. for 10 seconds and the rod expanded quickly into the hole, forming a tight interference with the cavity walls. The suture was clamped 6 to a spring balance and a force of 180N 7 was reached just before the sutures broke. The expanded rod did not come out and it was concluded that the relaxation of shape memory anchor was responsible for the tight fit.

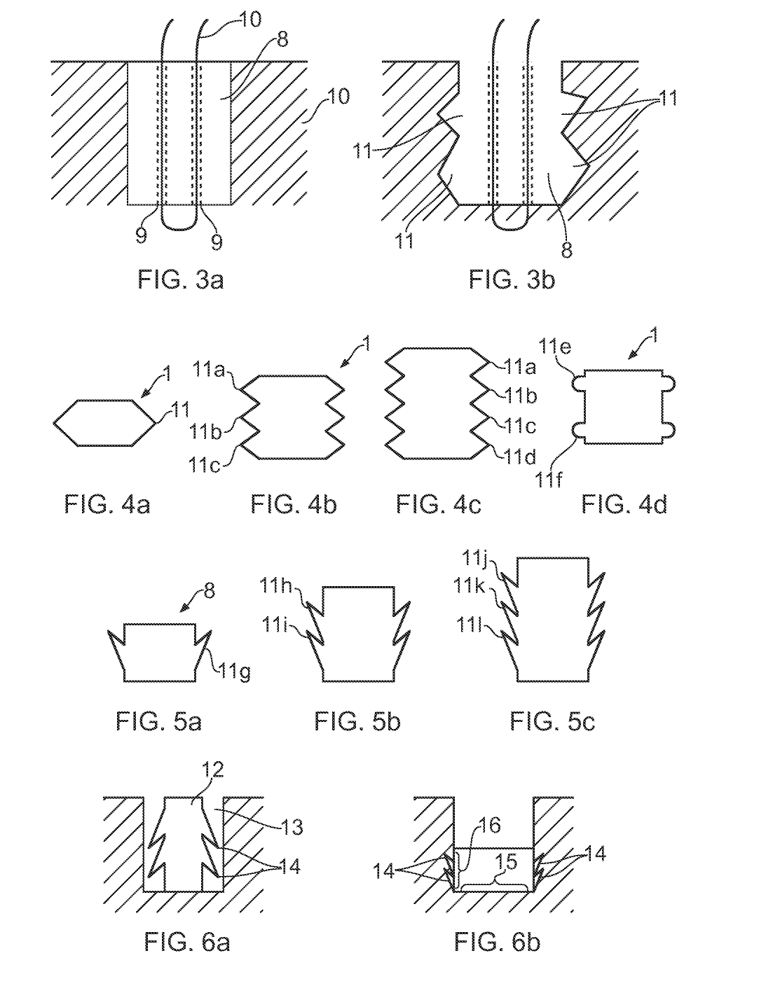

FIG. 3a illustrates a cylinder 8, made of shape memory material, with a single or double central hole or holes 9 through which a suture is fed 10. The anchor is inserted into a pre-drilled hole in the bone 10 with a press fit. Upon heating, the cylinder 8 will shorten along its y-axis, the central hole(s) will shrink in diameter and the outside edges of the cylinder will “accordion” resulting in circumferential ribs 11 thus digging into the surrounding bone and providing an interference fit, increasing the interfacial resistance and providing a solid anchor as demonstrated in FIG. 3b. FIG. 3b illustrates the suture anchor once the shape memory material has been activated.

FIG. 4 illustrates embodiments of the present invention comprising accordion shapes that have a single rib 11 as shown in FIG. 4a, a triple rib 11a, 11b and 11c as shown in FIG. 4b or a quadruple rib 11a, 11b, 11c, and 11d as shown in FIG. 4c.

It is envisaged that other embodiments may comprise between 1 and 10 ribs. The ribs may be either pointed or curved, the curved embodiment being shown in FIG. 4d. The ribs may be either continuous or non continuous along the length of the cylinder. The device may or may not comprise a central bore in these embodiments.

FIG. 5 illustrates a further embodiment which will, upon heating, have circumferential ribs (11g, 11h, 11i, 11j, 11k,) that point upwards in a similar manner to a barb (see FIG. 5a). As above, the central hole will close as the length of the cylinder decreases. The number of circumferential barbs can be between 1 and 10 (FIG. 5b-5c).

A further embodiment is illustrated in FIG. 6a which comprises an anchor 12 that levers itself into the drill hole 13. The anchor will initially have downward pointing barbs 14 on its surface which fit into the drill hole as part of the total diameter of the anchor.

FIG. 6b illustrates the embodiment of FIG. 6a upon activation e.g. by heating in which the diameter 15 of the anchor increases and the length 16 decreases. At the same time, the barbs 14 flip through ninety degrees to secure the anchor in place, but also to drive the anchor into the full depth of the drill hole, ensuring there is no gap behind it. The number of barbs can vary between 1 and 10.

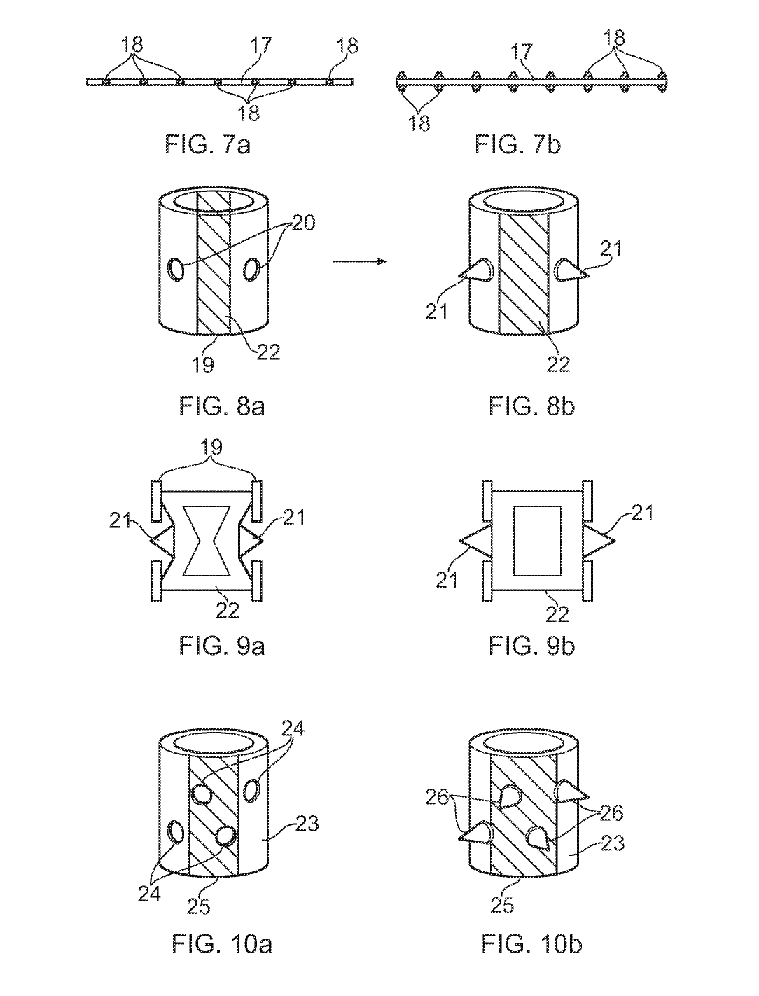

FIG. 7a illustrates a shape memory polymer suture thread 17. Upon heating, small sections 18 of the thread will contract to elicit either pointed or round circumferential ribs along the length of the suture. FIG. 7b illustrates the suture thread following activation e.g. by heating. The ribbing increases the grip and stability of the suture in the tissue. The ribbing may be barbed for example.

FIGS. 8a and 8b illustrate a device of an embodiment of the present invention to effect fixation by utilising the shape changing nature of SMPs. An SMP anchor tube 19 has holes 20, from which spikes 21 emerge following activation of the SMP material structure 22.

FIGS. 9a and 9b shows a detailed cross sectional view of the device illustrated in FIG. 8. The spikes 21 may be continuous with the SMP and composed of the SMP material or may be a different material physically connected to the SMP structure 19. When activated, as shown in FIG. 9b, the SMP structure 22 undergoes a vertical contraction, causing straightening of bent components, to force spikes 21 out of the device 19. This allows insertion, subsequent activation of the SMP 21 and thus enhanced fixation. Spikes which emerge due to SMP transformation may be used in multiple axes around the circumference of the device and/or numerous times along the length of the device.

FIGS. 10a and b illustrate a device of an embodiment of the present invention to effect fixation by utilising the shape changing nature of SMPs. The device has holes 24, from which spikes 26 emerge following activation of the SMP structure 25.

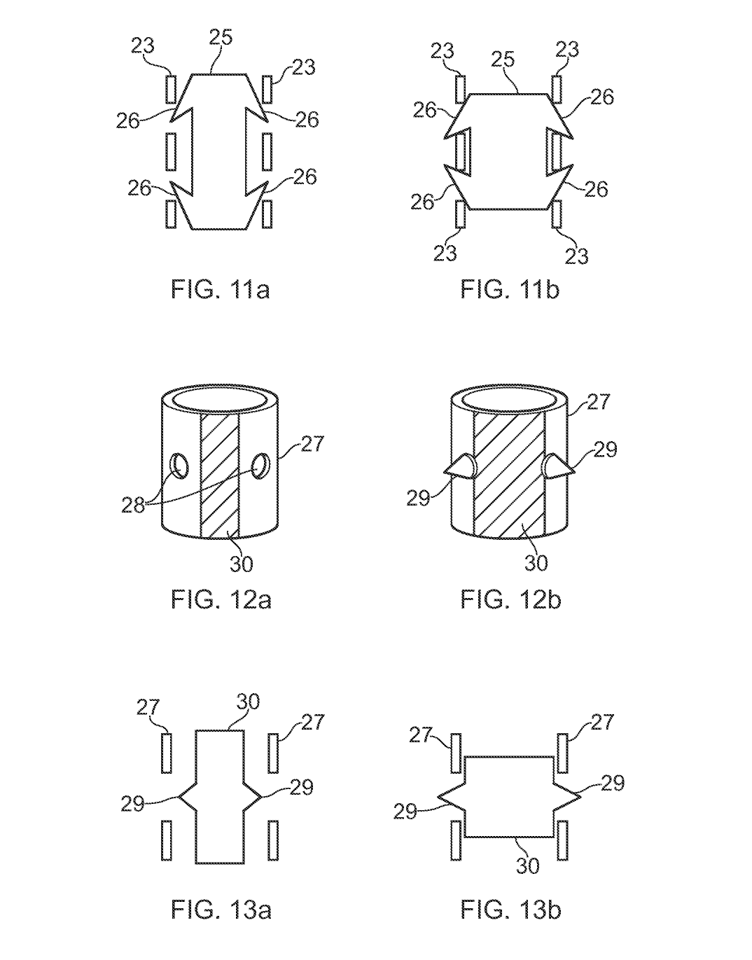

FIG. 11 is a cross sectional view of the device envisaged in FIG. 10. The pre-activated form of the device is shown in FIG. 11a. The spikes 26 may be continuous with the SMP and composed of SMP or may be a different material physically connected to the SMP structure 25. When activated, as shown in FIG. 11 b, the SMP structure 25 undergoes a vertical contraction, causing shortening/widening of the SMP component 25, to force spikes 26 out of the device 23 to allow insertion, subsequent activation of the SMP 25 and thus enhanced fixation. Spikes which emerge due to SMP transformation may be used in multiple axes around the circumference of the device and/or numerous times along the length of the device.

FIG. 12 illustrates a device of an embodiment of the present invention to effect fixation by utilising the shape changing nature of SMPs. FIG. 12a shows a device pre-activation An SMP anchor tube 27 has holes 28, from which spikes 29 emerge following activation (as shown in FIG. 12b) of the SMP structure 30.

FIGS. 13a and 13b show a cross sectional view of the device depicted in FIG. 12. The spikes 29 may be continuous with the SMP and composed of SMP or may be a different material physically connected to the SMP structure 30. When activated, as shown in FIG. 13b, the SMP structure 30 undergoes a vertical contraction, causing shortening/widening of the SMP component 30, to force spikes 29 out of the device 27. This will allow insertion, subsequent activation of the SMP 30 and thus enhanced fixation. Spikes which emerge due to SMP transformation may be used in multiple axes around the circumference of the device and/or numerous times along the length of the device.

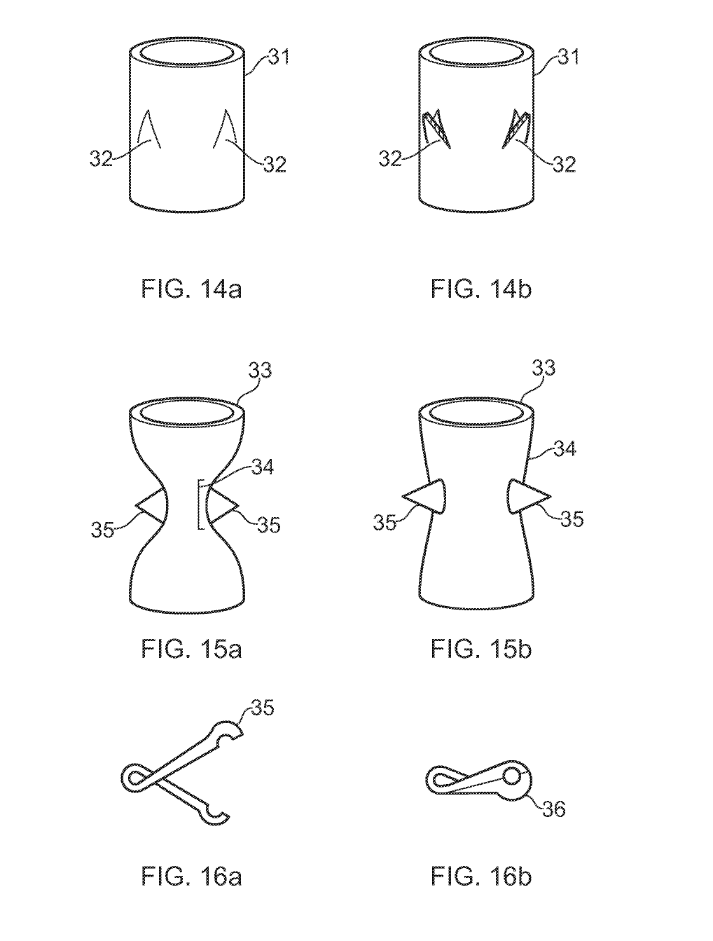

FIGS. 14a and 14b illustrate a device to effect fixation by utilising the shape changing nature of SMPs. An SMP anchor tube 31 has fins which are folded in 32, in the unactivated (FIG. 14a) state, but these unfold (FIG. 14b) when activated to effect enhanced fixation. Folded fins which emerge due to SMP transformation may be used in multiple axes around the circumference of the device and/or numerous times along the length of the device.

FIGS. 15a and 15b illustrate a device to effect fixation by utilising the shape changing nature of SMPs. An SMP anchor tube 33 has thinner orientated sections 34 along the length. The thin sections 34 have spikes 35 mounted on them to allow insertion. The spikes 35 may be continuous with the SMP and composed of SMP or may be a different material physically connected to the SMP structure 34. When activated, as shown in FIG. 15b, the SMP structure 34 undergoes a vertical contraction, causing shortening/widening of the SMP component 35, to force spikes 35 radially outward to allow insertion, subsequent activation of the SMP 35 and thus enhanced fixation. Spikes which emerge due to SMP transformation may be used in multiple axes around the circumference of the device and/or numerous times along the length of the device.

A device to effect a closure action is shown in FIGS. 16a and 16b. FIG. 16a shows the device pre-activation. The SMP clip component 35 is activated (as shown in FIG. 16b) to effect closure 36 of a clip device to cause a clipping (or pinching) action on activation of the SMP.

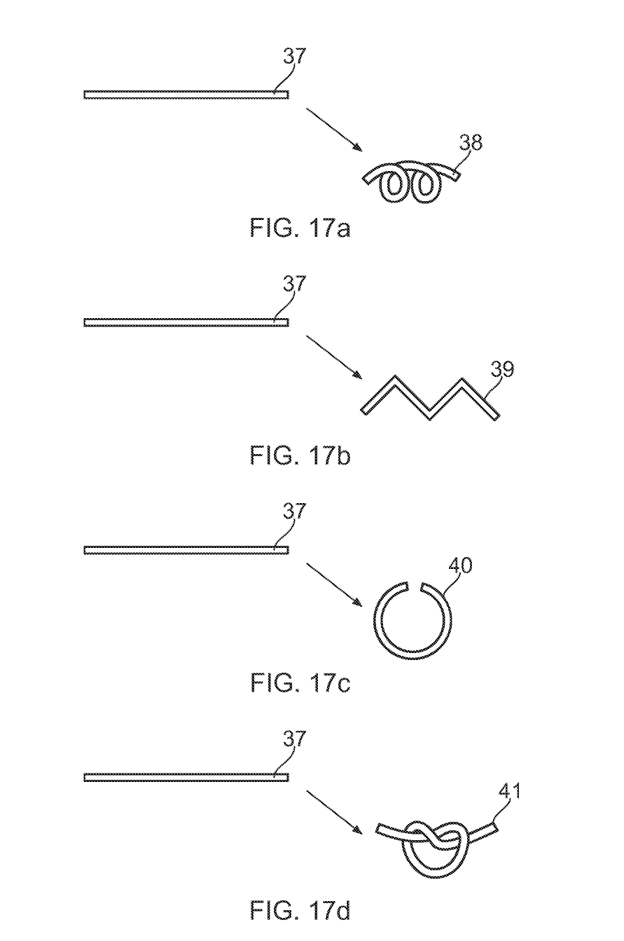

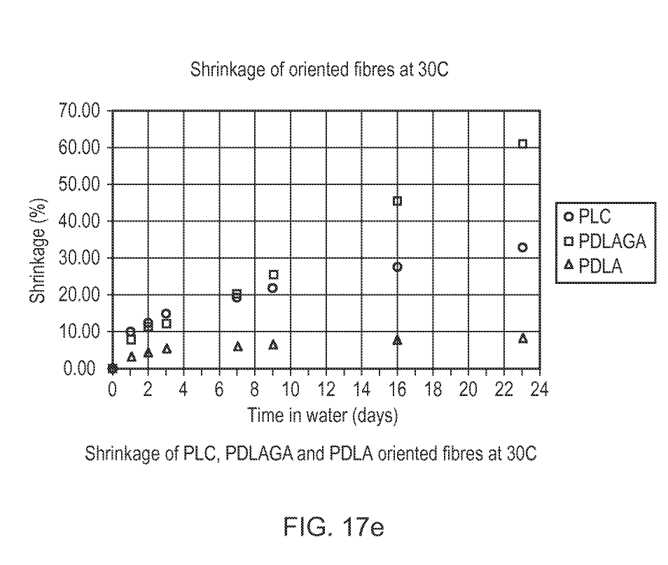

FIGS. 17a to 17d illustrate various embodiments comprising a range of devices to effect fixation by utilising the shape changing nature of SMPs. A pin 37 (which has a geometry with a regular cross section, or alternatively a cross section which tapers to a point such as a needle) is shown in FIGS. 17a to 17d. On activation, the device transforms geometry from pin 37 to a helical structure 38 (FIG. 17a); a planar zig-zag structure 39 (FIG. 17b), a loop structure 40 (FIG. 17c) or a knot structure 41 (FIG. 17d).

This device may also be used to effect closure to bring tissues together. Amorphous poly (D,L lactide-co-glycolide) (PDLAGA) with 35% w/w CaCO₃ (PLC), PDLAGA and poly(D,L lactide) (PDLA) fibres were prepared using a twin screw extruder. The fibres were drawn using the zone drawing technique in which the fibre is pulled at constant force through a local heater at 60° C.

FIG. 17e illustrates the shrinkage properties of orientated fibres at 30° C. PDLAGA, PLC and PDLA drawn fibres were immersed into water at 30 C and 37 C. In FIG. 17e, it is shown that at 30° C. after 9 days the shrinkage of PLC and PDLAGA are very similar, but at 16 days and thereafter PDLAGA shrinks and swells more than PLC. PDLA at 30° C. only shrinks about 6% after 23 days. At 37° C. PLC, PDLAGA and PDLA drawn fibres shrink completely after 1 day in water at 37 C, recovering the dimensions of the undrawn fibres.

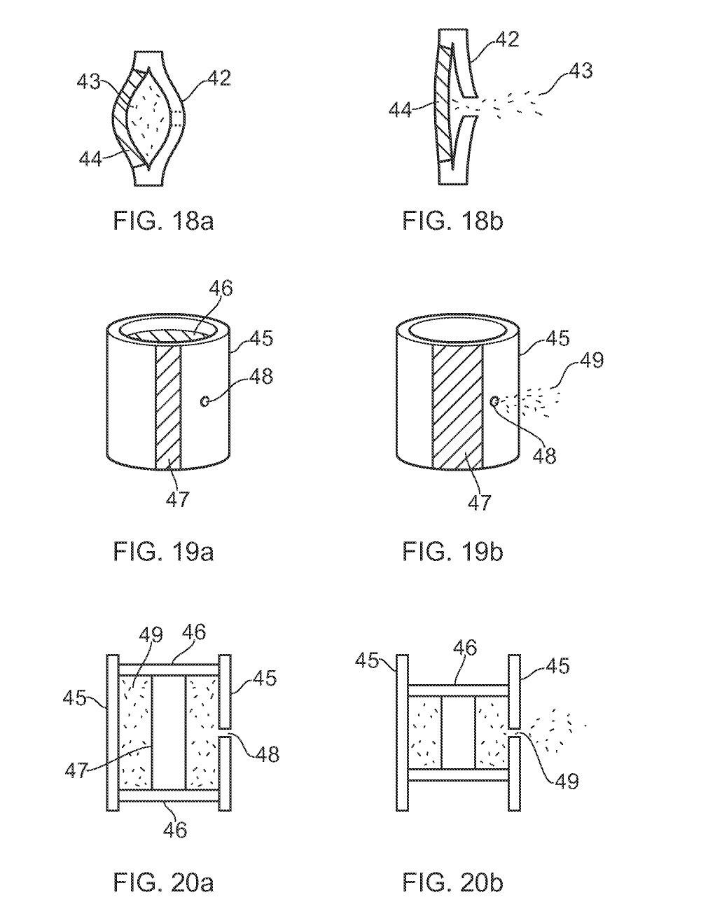

FIGS. 18a and 18b illustrate a device to deliver a fluid upon activation by utilizing the shape changing nature of SMPs. A device 42, shown in a pre-activation form in FIG. 18a, comprises a vessel for fluid 43 acts to deliver the fluid on activation of the SMP 44 (as shown in FIG. 18b), by causing a contraction of the internal volume of the device. The fluid may be a cement, drug, curing agent, material repair agent, antibiotic etc. A thin membrane may be used to prevent premature delivery of fluid. The expulsion of a fluid may be used as a cement or glue to effect fixation.

FIGS. 19a and 19b illustrate a device to deliver a fluid upon activation. A device 45 comprising a vessel for fluid acts to deliver the fluid 49 through a designated release point 48 on activation of the SMP component 47 in combination with a restricting end piece 46. The fluid may be a cement, drug, curing agent, material repair agent and/or antibiotic. A thin membrane may be used to prevent premature delivery of fluid. FIG. 19a illustrates a pre-activation form and FIG. 19b illustrates the device on activation of the SMP material.

FIG. 20a and FIG. 20b show a cross sectional view of the device of FIG. 19. The fluid 49 is contained within a cavity enclosed by the walls of the device 45, and end pieces 46 which may be continuous with the SMP component 47 or made of non-SMP material but physically connected to the SMP component 47.

On activation (shown in FIG. 20b) of the SMP component 47, the SMP component 47 reduces length, and increase thickness, bringing end pieces 46 closer together and causing fluid 49 to be expelled through orifice 48. A thin membrane may be used to prevent premature delivery of fluid through the orifice 48. The expulsion of a fluid may be used as a cement or glue to effect fixation.

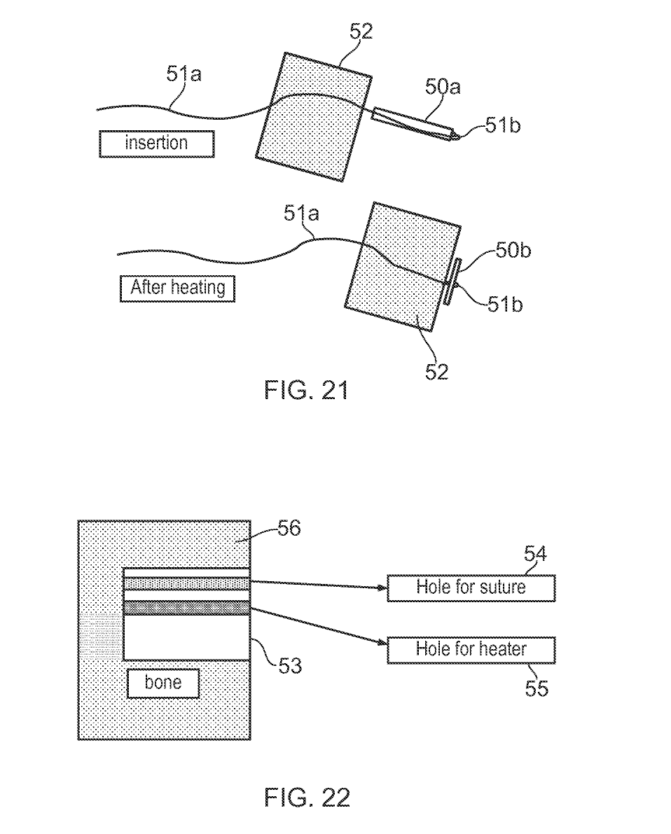

FIG. 21 illustrates a thin die-drawn polymer sleeve 50a which is placed over a suture 51a. The internal diameter is approximately the same as the suture diameter. A knot 51b is provided in the distal end of the suture. Optionally the distal end of the sleeve is tapered to aid penetration into tissue. The sleeve and suture are pushed through the tissue 52, probably, but not necessarily, through a pre-formed hole until the sleeve clears the tissue. Heat is then applied to the sleeve when the suture is in tension and the sleeve polymer relaxes forming a washer 50b which will securely stop the knot from being pulled back through the tissue. This forms an anchor for the suture to be used to hold parted tissue to the first tissue.

FIG. 22 illustrates a plug of die-drawn polymer 53 with two holes (54, 55) for use as a suture anchor into bone 56 tissue. The first hole 54 is off-centre in the plug and has a suture passed through it and tied off in a knot. The second hole 55 is for insertion of a heater tool to permit the polymer to ‘relax’ and expand to form a secure fastening into the hole. The suture is then anchored securely into the bone.

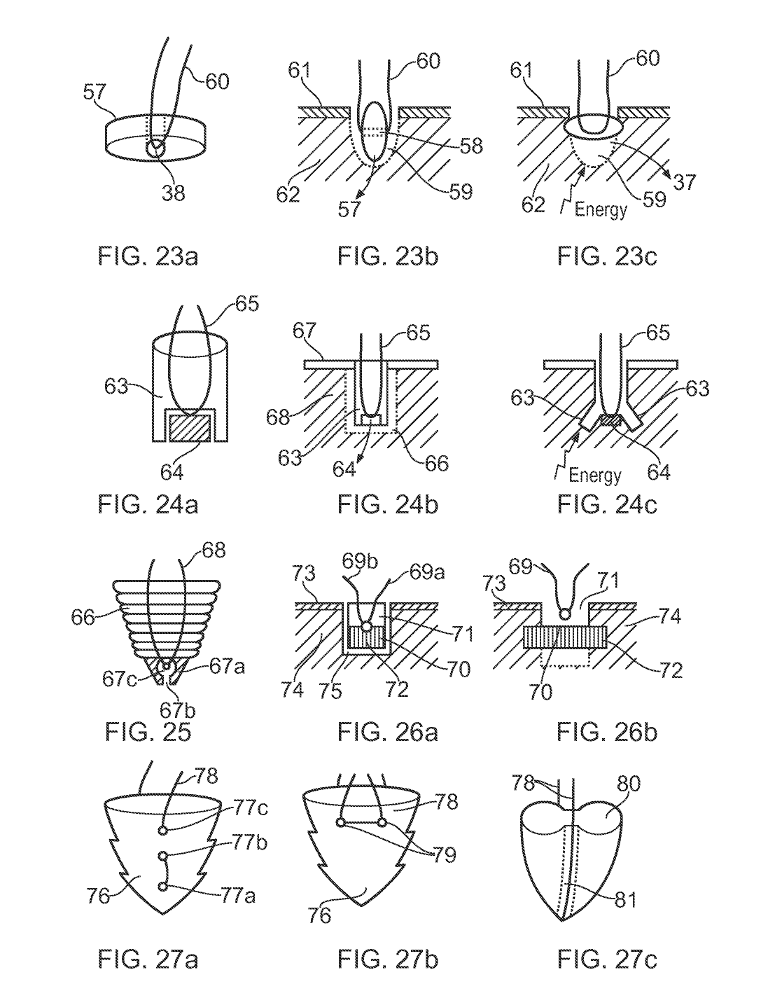

FIG. 23 illustrates a suture anchor which is formed from a round SMP billet by forming it into a long anchor when drawn. This can be released into a thick cylinder shaped material upon activation by an appropriate stimulus. FIG. 23a depicts an oval shaped suture anchor 57 with a centrally orientated hole 58 which accommodates the suture material 60. The device can be inserted into a prepared anchor site (FIG. 23b), which can be an orthopaedic site with cortical 61 and cancellous bone 62 containing a hole 59. The device 57 is deployed by inserting it vertically into the pre-prepared hole 57, with the suture material 60 running through the device 58. Upon activation (shown in FIG. 23c), the anchor device 57 flips into a longitudinal direction and forms a circular disc shape fixating the suture 60 into the anchor site 59.

Alternatively, anchor devices can be modified with shape memory materials to aid fixation in 2a site. FIG. 24a depicts a pronged device 63 which can be composed of either shape memory material or non-shape memory material, a suture 65 and an additional activation aid 64. This activation aid can be composed of a shape memory material and can be in an orientation or shape. FIG. 24b illustrates an example orthopaedic site with cortical 67 and cancellous bone 68, with a pre prepared anchor site 66 containing the exemplary device 63. A suture 65 can be threaded under the device 63. Upon activation FIG. 24c the device prongs 63 move outwards. This is assisted by the activation aid 64 which can be composed of shape memory material and relaxes in the longitudinal direction forcing the device prongs further.

Shape memory materials can also be used to aid the fixation of the suture material within the device. FIG. 25 shows a threaded anchor device 66 with a shape memory component 67a. The suture material 68 is fed through the small gap 67b in the shape memory component 67a into a wider cavity 67c. The threaded device is screwed into place then the shape memory component 67a is activated, fixating the suture material firmly in place.

Anchors can be manufactured with alternative stressed components which allows for the tailoring of material properties to aid fixation. FIG. 26a depicts a shape memory device 70 with two different stressed material components; area 71 a low stress area and area 72 a high stress area. The device 70 is inserted in a pre-prepared anchor site 75 within a cortical 73 and cancellous 74 bone structure, with a suture material 69a threaded through a device hole 69b. Upon activation, as shown in FIG. 26b, the high stress portion 72 of the device 70 deforms so that the final diameter increases fixating the device with the cancellous bone 74. The lower stress portion 71 also deforms to fixate within the cortical bone 73.

Shape memory anchors can be modified geometrically or physically to accommodate suture materials. FIG. 27a shows a shape memory anchor 76 with multiple holes 77a-c in the vertical direction to accommodate a figure of eight 78 suture configuration. Alternatively the holes 79 can be in the longitudinal direction as depicted in FIG. 27b to accommodate alternative suturing configurations 78 in the device 76.

Alternatively the geometry of the shape memory device can be modified as to accommodate the suture materials, as shown in FIG. 27c. The tapered oval device 80 has central grooves 81 machined out so the suture material 78 can be fixated within these following activation.

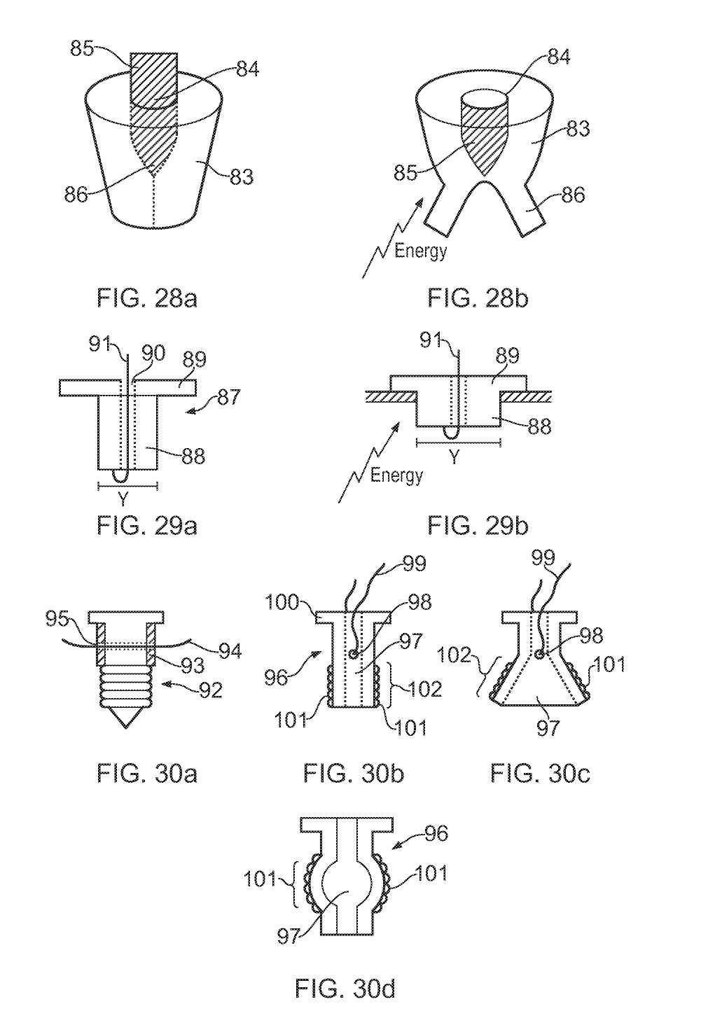

Shape memory anchors can be also utilised to fixate pins and other orthopaedic devices within the body. FIG. 28 illustrates a shape memory anchor 83 with a split prong configuration 86 and a tapered hole 84 for receiving a pin 85 or other device. FIG. 28b illustrates an activated device where the pin 85 has been pushed into the central tapered hole 84. The devices fixation prongs 86 relax in the longitudinal direction and the pin 84 has been fixated within the device 83.

Fixation devices such as tacs and pins can also be constructed from shape memory materials with various properties to enable enhanced fixation. These anchors can also be used in conjunction with other orthopaedic devices such as sutures and plates. FIG. 29a depicts a shape memory tac 87 which has an SMP portion 88 and a non-SMP head. The tac can optionally contain a hole 90 for threading sutures 91 or other devices through. Upon activation (FIG. 29b) the SMP portion's (88) width (y) becomes wider and the shaft shorter, thus fixating the device with the application site. The non-SMP head portion 89 retains its original geometry and fixates the device on top of the surface.

Fixation screws such as those depicted in FIG. 30a can be modified with SMP to aid fixation of both the implant and additional intrinsic devices such as sutures. The threaded screw 92 contains a shape memory collar 93 running the circumference of the device 93, with an optional hole 95 running longitudinally through the centre of the device to house the suture material 94. Upon activation the shape memory collar 93 grips the suture 94 and fixates the device 92 in place.

In addition FIG. 30b to d shows additional examples of tac fixation devices modified with shape memory material to aid fixation. FIG. 30b shows a tac fixation device 96 with an internal shape memory component 97 which contains a hole 98 for a suture material 99. The device also contains a head portion 100 with additional fixation aids 101 positioned on the lower half of the tac 102. Upon activation (as shown in FIG. 30c) the lower half 102 of the shape memory portion 97 relaxes forcing it outwards and engaging the fixation aids 101 into the surrounding tissue. The suture hole 98 also constricts fixing the suture 99 in place. The tac can also have shape memory portions along alternative lengths of the device as shown in FIG. 30d. The device 96 contains a shape memory portion 97 in the central region 102 of the device with fixation aids 101 also positioned within this area. Upon activation the shape memory portion 97 relaxes in the longitudinal direction forcing the fixation aids 101 in an outward direction.

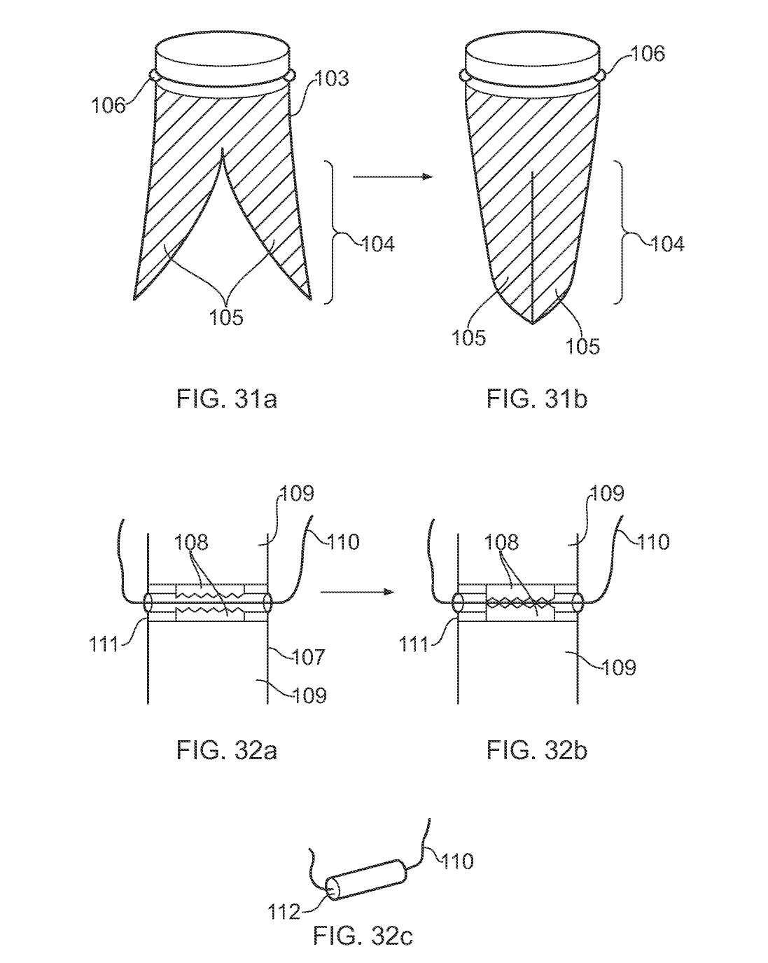

Various processing methods can be used to generate shape memory devices. FIG. 31a depicts an injection moulded shape memory device 103 with two forked prongs 105 situated in the bottom half of the device 104 and a suture hole 106. This open position device is cold pressed forcing shape memory properties into appropriate regions of the device. FIG. 31b shows the cold compressed device with the forked prongs 105 situated within region 104 device in a closed position. The forks 105 have the shape memory properties and will expand outwards upon activation by an appropriate stimulus.

Shape memory materials may be used as a locking mechanism within existing devices to fixate various items or to initiate a change in another material. FIG. 32a depicts a shape memory anchor device 107, with shape memory portions 109 and gripping member 108 within the shape memory portions 109. The suture material 101 is passed through the gripping member 108 and within the gripping member containment zone 111. Upon activation (as shown in FIG. 32b) the shape memory portions 109 relax causing the gripping member 108 to fixate the suture 110 within gripping member containment zone 111. Alternatively the gripping member 108 can be solely composed of shape memory material within a non shape memory device. Upon activation the gripping member 108 will relax fixating the suture in place within the device. Shape memory tubes may also be used in combination with sutures to fixate them within an appropriate surgical site. FIG. 32c shows a SMP tube 112 with a suture 110 running through the centre.

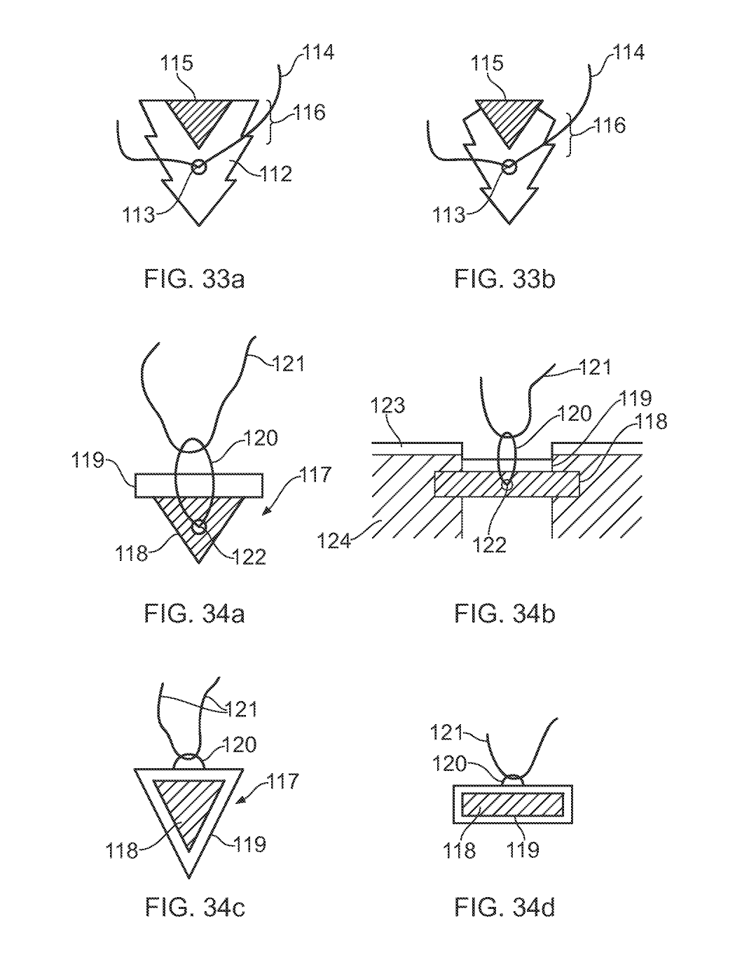

Shape memory may also be used to activate non-shape memory devices. FIG. 33b shows a pre-activation barbed anchor device 112 with an eyelet 113, a suture material 114 running through the eyelet 113, and a shape memory portion 115 contained within a particular segment of the device 116. Upon activation (as shown in FIG. 33a) the shape memory portion 115 relaxes and forces the top segment of the device 116 outwards causing fixation.

If a shape memory device moves during relaxation it may result in a loss in tension of the suture material. FIG. 34a depicts a device which overcomes the problems associated with the loss of tension and positioning of suture materials. The device 117 contains a shape memory portion 118 and a shape memory/non-shape memory portion 119 with an eyelet 122, and a suture attachment region 120 threaded through the eyelet 122. The suture 121 is threaded through the suture attachment region 120. The device is then inserted into a pre-prepared site (FIG. 34b) in cortical 123 and cancellous bone 124. Upon relaxation the shape memory portion 118 forms a bar structure fixating and tensioning the suture 121 through the suture attachment region 120 into the device.

FIG. 34c depicts an alternative embodiment where the device 117 is composed of a shape memory material 118 with an outer skin composed of a non-shape memory skin 119. The device also has an additional suture attachment region 120 which allows for a suture material 121. Upon relaxation (FIG. 34d) the shape memory portion 118 relaxes in a longitudinal direction forcing the suture 121 to be tensioned and positioned within the suture attachment region 120.

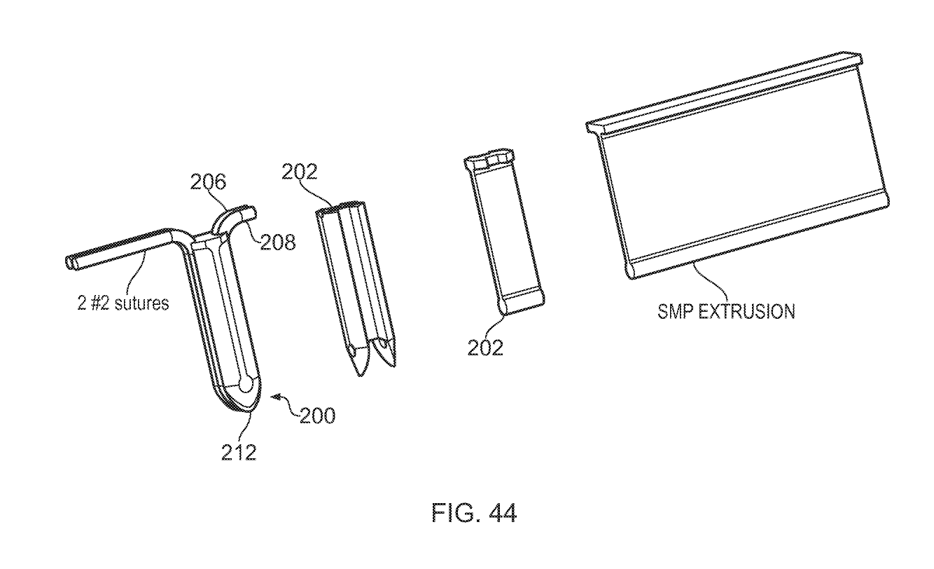

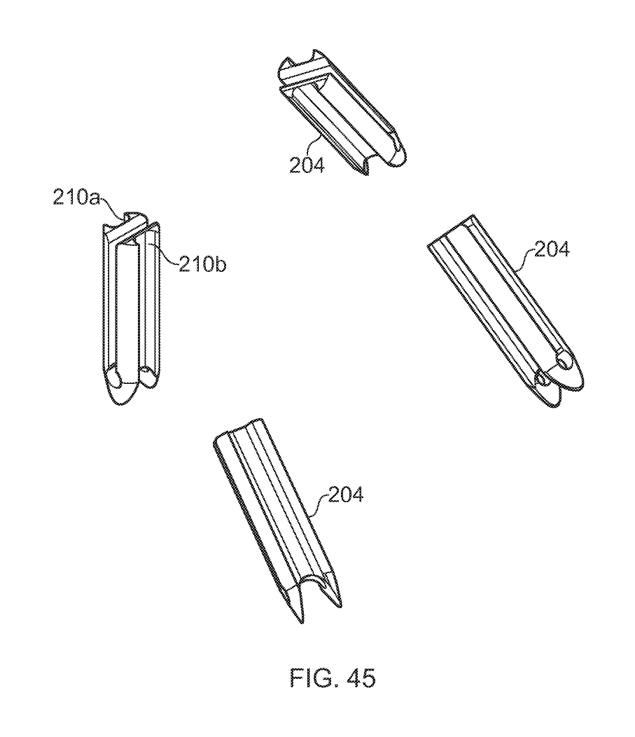

FIG. 44 and FIG. 45 illustrate an embodiment of the present invention, a device 200 which comprises an SMP material portion and a non-SMP material portion. In particular, FIG. 44 illustrates a schematic representation of a process used to make the device 200. The device 200 is formed from an SMP material component 202 and a non-SMP component 204 e.g. moulded plastic. The non-SMP component 204 includes one or more grooves 210a, b on an outer surface thereof which are sized to accommodate one or more sutures 206,208. The sutures pass down the length of the groove on a first lateral surface, around the distal end 212 of the anchor and up the opposing lateral surface. In use, the SMP material component is activated, causing radial expansion of the SMP material component, and causing the sutures to be held against the surface of the cavity in which the anchor is placed.

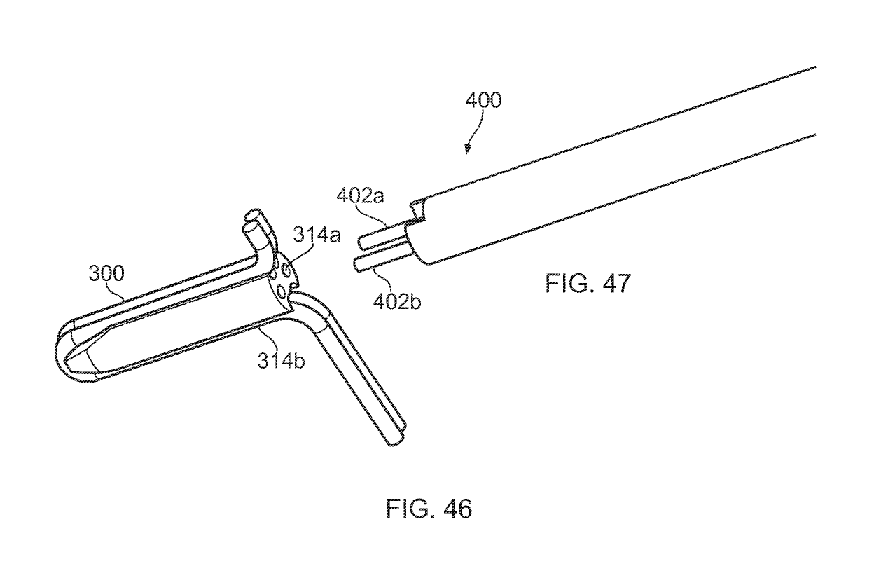

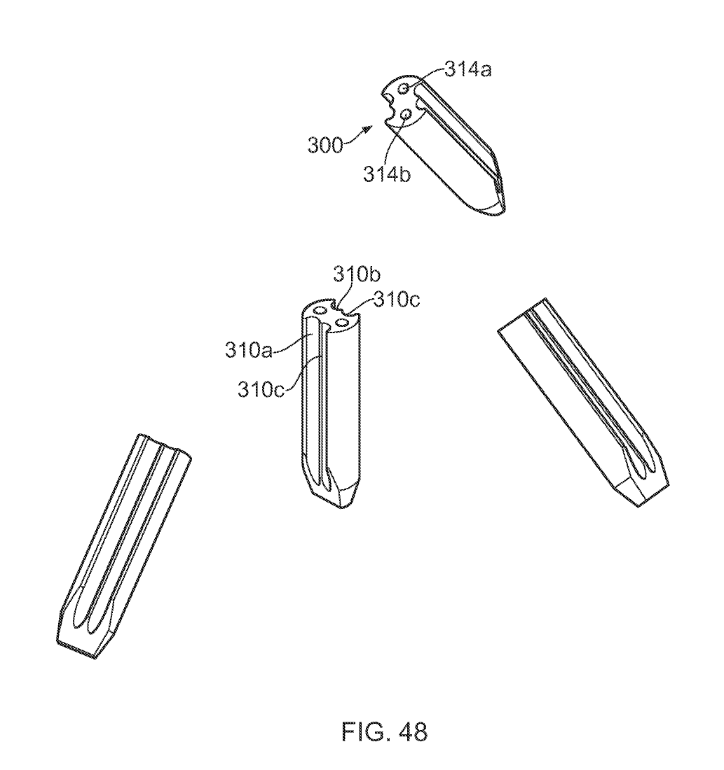

FIG. 46 illustrates a further embodiment which comprises a suture anchor device 300 which is composed entirely of an SMP material. The device 300 includes one or more grooves 310a, b, c, d on its outer surface which are sized to accommodate a suture as described above. The device 300 includes one or more central channels 314a, b to accommodate the guide rods of the tool 400.

FIG. 47 illustrates a tool 400 for aiding insertion of the suture anchor illustrated in FIG. 46. The tool 400 includes a pair of guide rods 402a, b which fits into the channels 314a, b of the device to aid insertion. The tool also includes a heater, which may be supplied by the guide rods.

### EXAMPLES

### Example 1

### Hybrid Anchor Prototype 1

To produce a rod for die-drawing, 500 g of poly(DL-lactide-co-glycolide) (PDLGA) 85:15 supplied by Purac Biomaterials was vacuum dried at 50° C. for 3 days. The dried polymer was stored in sealed bags containing desiccant sachets until needed. The polymer was then extruded using a Prism extruder with a 3 mm die, air-cooled haul-off belt, caterpillar haul-off with an air-cooling ring placed between the belt and caterpillar haul-offs. The polymer was fed to the extruder at 750 g/hr using a computer controlled pellet feeder and a screw speed of 225 rpm. The extrusion conditions used are shown below.

| | Heater zones (° C.) | | | | | Pressure | Torque |
|---|---|---|---|---|---|---|---|
| | Inlet | 1 | 2 | 3 | Die | Bar | Nm |
| Set | 0 | 150 | 180 | 190 | 190 | — | — |
| Recorded | 95 | 156 | 187 | 198 | 188 | 19-20 | 8.6-9.9 |

The haul-off and belt speeds were varied to produce rod diameters between ˜3.5 and 1 mm. The rod was chopped in to 0.5 to 1 m lengths as it emerged from the caterpillar haul off. The rods were packed in a plastic tube with desiccant and placed in a freezer.

Shape-memory polymer rod was then produced by die-drawing. Die-drawing of rod produced above was carried out by pulling the rod through a heated die fitted to an Instron 5569 Universal Testing Machine fitted with a 1 kN load cell. The die had a 1.5 mm diameter and was controlled at a temperature of 65° C. The diameter of the rod pre-drawing was 3.17 mm and post-drawing 1.40 mm, giving a draw ratio (final length/initial length) of 5.13.

Draw ratio was calculated as:

(Pre-draw diameter)²/(Post-draw diameter)²

The shape-recovery properties of this rod were tested by heating either in air at 80° C. or in water at 37° C. Sample lengths were measured periodically until there was no further change in shape.

The recovery ratio and % shape recovery were calculated as follows:

Recovery ratio=Pre-recovery length/Post recovery length

$$\% Shape Recovery = \frac{Pre \text{-} re covery length - Post recovery length .}{Pre \text{-} recovery length - ( Pre \text{-} recovery length / Draw ratio )} \times 100$$

In air at 80° C. the rod had a Recovery Ratio of 4.5 and a % Shape Recovery of 96.6%. In water at 37° C. it had a Recovery Ratio of 4.32 and a % Shape Recovery of 95.5%.

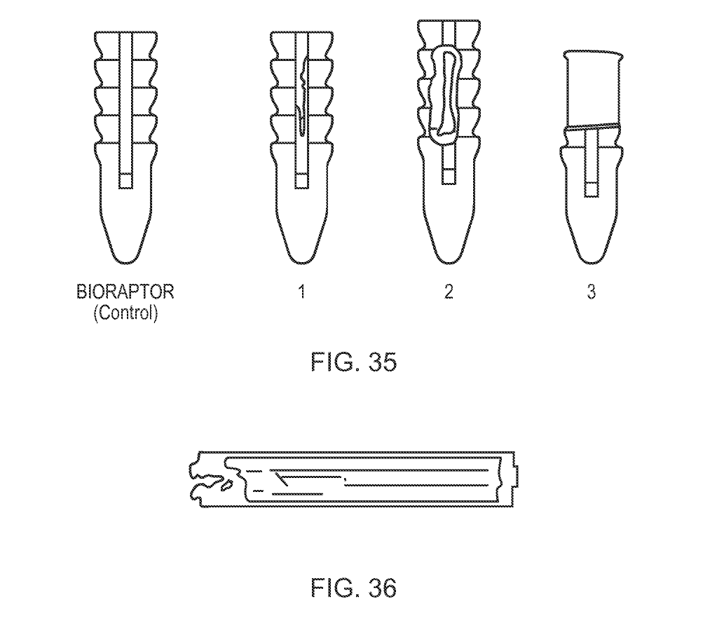

A hybrid SMP anchor was produced by modification of a standard PEEK anchor (BIORAPTOR 2.3PK, produced by Smith & Nephew). 5.5 mm deep holes 1.6 mm in diameter were drilled in the ends of the anchors, which were then cut on both sides for the full length of the hole. 5 mm lengths of the 1.4 mm diameter SMP rod were then fitted into the hole. This was labelled Prototype 1—see FIG. 35.

### Example 2

### Hybrid Anchor Prototype 2

The dried PDLGA of Example 1 was moulded to produce rods approximately 6 mm in diameter by 55 mm long using a Haake MiniJet Injection Moulder. The moulding conditions were:

| | |
|---|---|
| Cylinder | 190° C. |
| Mould | 40° C. |
| Injection Pressure | 800 bar for 10 seconds |
| Post Pressure | 450 bar for 5 seconds |

To produce a shape-memory strip from this rod, the rod was compressed in a press. The moulded rod was heated in an oven between metal plates at 50° C.; 0.8 mm shims were also placed between the plates to set the desired thickness of the SMP. The plates were then removed from the oven, transferred to a hydraulic press with platens cooled to below 20° C.

The press was then closed immediately and pressure of 200 kN applied before the plates or rod had cooled, once the plates and SMP strip product had cooled, the press was opened and SMP strip removed.

A maximum deformation ratio for the SMP strip was calculated as follows:

$$Maximum deformation ratio = \frac{Original diameter \times Original length}{Strip thickness \times Strip length}$$

The initial rod had a diameter of 5.35 mm and a length of 54.52 mm; the SMP strip had a thickness of 1.22 mm and a length of 60.66 mm, giving a deformation ratio of 3.94.

To produce a hybrid anchor using the SMP strip, a 1.3 mm wide slot was cut into a BIORAPTOR 2.3PK device and a cut was then made from the top of the slot to the end of the anchor. The slot was filled with an SMP strip cut from the middle of the moulded sheet to fit. This was labelled Prototype 2—see FIG. 35.

### Example 3

### Hybrid Anchor Prototype 3

Injection moulded rods as described in Example 2 were die-drawn as described in Example 1 except that a 3 mm or 2.75 mm die was used and the die temperature was 55° C. or 58-62° C. respectively. The initial diameter of the rods was 5.25 mm and the final diameter was either 2.9 mm (for the 3 mm die) or 2.7 mm (for the 2.75 mm die).

The 2.9 mm samples had a mean draw ratio of 3.21. The recovery properties were measured in water at 37° C. as described in Example 1 and the rods were found to have a shape recovery of 99.1%.

The 2.7 mm samples had a mean draw ratio of 3.79. The recovery properties were measured in water at 37° C. as described in Example 1 and the rods were found to have a mean recovery ratio of 3.4 and a mean shape recovery of 95.41%.

The die-drawn SMP rods were cut to lengths of 4.5 mm. The end 4.5 mm was removed from BIORAPTOR 2.3PK anchors and replaced by a piece of SMP rod to produce a hybrid anchor. This was labelled Prototype 3—see FIG. 35.

### Example 4

### 2.7 mm Diameter SMP Rod Prototypes

The die-drawn 2.7 mm rods described in Example 3 were used. The rods were cut to the same length as the BIORAPTOR™ control anchors (11.5 mm) and a slot was made at one end to accommodate a suture (FIG. 36).

### Example 5

### 1.9 mm Diameter SMP Rod Prototypes

A PDLGA 85:15 rod with diameter 3.3 mm was produced by extrusion as described in Example 1. The rod was then die-drawn as in Example 1 except that a 2 mm die was used with a drawing temperature of 60° C. The die-drawn SMP rod had a final diameter after drawing of 1.9 mm and a draw ratio of 2.99. The recovery properties were measured in water at 37° C. as described in Example 1 and the rods were found to have a mean shape recovery of 98.5%.

The rods were cut to the same length as the BIORAPTOR™ control anchors (11.5 mm) and a slot was made at one end to accommodate a suture (FIG. 36).

### Example 6

### 0.71 mm Diameter SMP Rod Prototypes

PDLGA 85:15 rod with diameter 1.57 mm was produced by extrusion as described in Example 1. The rod was then die-drawn as in Example 1 except that a 0.75 mm die was used with a drawing temperature of 60° C. The die-drawn SMP rod had a final diameter after drawing of 0.71 mm and a draw ratio of 4.89. The recovery properties were measured in water at 37° C. as described in Example 1 and the rods were found to have a recovery ratio of 3.73 and a shape recovery of 92.0%.

The SMP rod was cut into 5 mm lengths and left unslotted.

### Example 7

### Pull-Out Testing of Hybrid Anchors in 10PCF “Sawbones™” Foam

The pull-out force was measured using an Instron 5569 fitted with a 1 kN load cell. The samples were tested in 10 PCF (pounds per cubic foot) solid rigid polyurethane foam (Sawbones AB, Sweden). The Sawbones foam was cut to produce strips with a 3×3 cm cross section to fit in a slotted aluminium support fitted in the lower Instron grip. Holes were drilled in the block using the BIORAPTOR drill (2.6 mm) and drill guide, or other drills, with a spacing of a minimum of 5×the diameter of the device. The hole dimensions used are shown in the table below.

TABLE 1
Hole dimensions used for pull-out tests.

| | Hole dimensions (mm) | | |
|---|---|---|---|
| Device(s) | Diameter | Depth | Spacing |
| Biroaptor 2.3 PK | 2.6 | 20 | 18 |
| Combination prototypes 1, 2 & 3 | | | |
| 2.7 mm diameter SMProds | | | |
| 1.9 mm daimeter SMProds | | | |
| Biroaptor 2.3 PK | 3.0 | 20 | 18 |
| Combination prototype 3 | | | |
| 2.7 mm diameter SMProds | | | |
| 0.71 mm diameter SMProds | 1.5 | 10 | 12 |
| | 1.0 | 10 | 12 |

Anchors were inserted into the Sawbones test blocks using the BIORAPTOR™ insertion tool, or for the devices that would not fit the tool, a stiff wire was used. For each experiment, four replicate samples were prepared and tested. For wet testing, the holes were filled with water before device insertion.

Control samples were tested immediately after insertion, shape memory test samples were incubated in water at 37° C. to activate shape recovery. The incubation time used was 40 hours for diameters between 2 and 3 mm, 24 hours for diameters less than 2 mm. All samples were allowed to cool to room temperature before testing.

The devices were pulled out of the block by the suture at a rate of 508 mm min⁻¹ (20 inches min⁻¹). The maximum load was recorded.

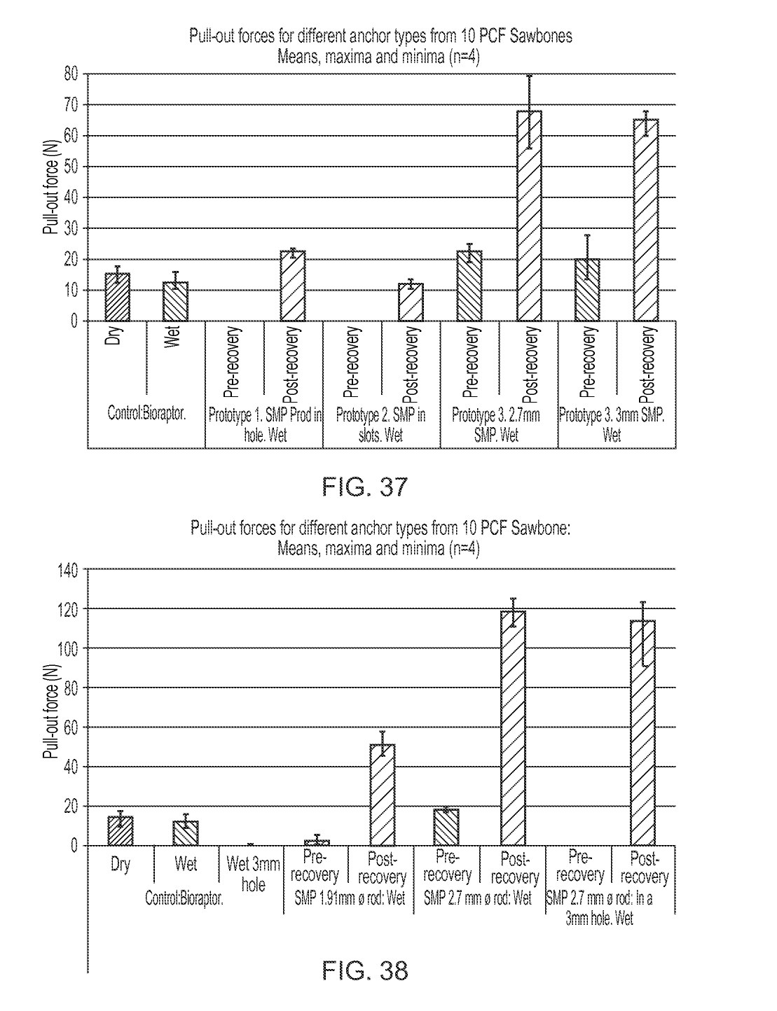

Table 2 and FIG. 37 show the pull-out test results for the hybrid anchor devices from 10PCF Sawbones foam. This material is a model for relatively poor quality, low density bone. The results show increased pull-out strength for the activated (post-recovery) SMP-hybrid anchor prototypes 1 and 3 compared to the BIORAPTOR™ control, especially Prototype 3, which had a >400% increase in pull-out force.

TABLE 2
Pull-out test results in 10PCF Sawbones, 2.6 mm holes

| | Test | Pull out force ( [symbol] ) | | | | | | | |
|---|---|---|---|---|---|---|---|---|---|
| Anchor used | Conditions | 1 | 2 | 3 | 4 | Mean | Max | Min | SD |
| Control: Bioraptor. | Dry | 18.0 | 18.0 | 13.7 | 12.5 | [symbol] | 18.0 | 12.5 | [symbol] |
| | Wet | [symbol] | 13.1 | 10.4 | [symbol] | 12.7 | [symbol] | 10.4 | 2.25 |
| Prototype 1. SMP rod in hole. Wet | Pre-recovery | | | | | | | | |
| | Post-recovery | [symbol] | [symbol] | [symbol] | [symbol] | [symbol] | [symbol] | [symbol] | 1.47 |
| Prototype 2. SMP in slots. Wet | Pre-recovery | | | | | | | | |
| | Post-recovery | [symbol] | [symbol] | 11.7 | 11.2 | 12.3 | 13.8 | 11.2 | 1.15 |
| Prototype 3. 2.7 mm SMP. Wet | Pre-recovery | 24.8 | [symbol] | [symbol] | 24.8 | 22.5 | [symbol] | [symbol] | [symbol] |
| | Post-recovery | 79.4 | 75.2 | 62.1 | [symbol] | [symbol] | [symbol] | [symbol] | 11.04 |
| Prototype 3. 3 mm SMP. Wet | Pre-recovery | [symbol] | 13.7 | 20.5 | 18.0 | 20.3 | 28.0 | [symbol] | 5.81 |
| | Post-recovery | [symbol] | [symbol] | [symbol] | [symbol] | [symbol] | [symbol] | [symbol] | 3.47 |

[symbol]

indicates data missing or illegible when filed

### Example 8

Pull-out testing of SMP rod devices in 10PCF “Sawbones” foam in standard and oversized holes.

Pull-out testing was carried out as described in Example 7 with the SMP rod devices. A 2.6 mm hole was used which was a “standard size” for the control BIORAPTOR™ and the 2.7 mm rod devices but was oversized for the 1.91 mm SMP rod anchor. A 3 mm hole was used as an oversized hole for the BIORAPTOR™ control and the 2.7 mm SMP rod anchor. The pull-out testing results are shown in Table 3 and FIG. 38.

TABLE 3
Pull-out testing of SMP rod anchors in 10PCF Sawbones in standard and oversized holes

| Anchor used | Test | Pull out force ( [symbol] ) | | | | | | | |
|---|---|---|---|---|---|---|---|---|---|
| ( [symbol] hole unless [symbol] otherwise) | Conditions | 1 | 2 | 3 | 4 | Mean | Max | Min | SD |
| Control: Bioraptor. | Dry | [symbol] | [symbol] | [symbol] | 12.5 | [symbol] | [symbol] | [symbol] | [symbol] |
| | Wet | [symbol] | 13.1 | 10.4 | [symbol] | 12.7 | [symbol] | 10.4 | 2.25 |
| | Wet 2 mm hole | 0.9 | 0.8 | 0.9 | [symbol] | 0.8 | 0.9 | 0.9 | 0.01 |
| SMP [symbol] mm [symbol] rods. Wet | Pre-recovery | [symbol] | 1.9 | 1.1 | [symbol] | [symbol] | 5.8 | 1.1 | 2.04 |
| | Post-recovery | [symbol] | 57.4 | [symbol] | 46.1 | [symbol] | 57.4 | 45.1 | 5.12 |
| SMP 2.7 mm [symbol] rods. Wet | Pre-recovery | 18.0 | 19.7 | 18.1 | 19.1 | [symbol] | 19.7 | [symbol] | 0.82 |
| | Post-recovery | 117.3 | 124.8 | 112.1 | 122.1 | 119.1 | 124.8 | 112.1 | [symbol] |
| SMP 2. 2.7 mm [symbol] rods in a 2 mm hole. Wet | Pre-recovery | | | | | | | | |
| | Post-recovery | 118.7 | 121.9 | 119.9 | 104.8 | 114.9 | 121.9 | 104.8 | 7.47 |

[symbol]

indicates data missing or illegible when filed

From the results shown in FIG. 38 it can be seen that an activated 2.7 mm SMP anchor has a >800% increase in pull-out strength compared to a similarly sized BIORAPTOR control, in a standard hole.

In the oversized hole the BIORAPTOR™ control does not have any significant pull-out-strength. On the other hand the 2.7 mm SMP rod has a very similar pull-out strength in both the standard and oversized holes.

The 1.9 mm rod in a 2.6 mm hole still showed a >300% increase in pull-out-strength compared to the BIORAPTOR control in the same sized hole.

### Example 9

Pull-out testing of hybrid anchor and SMP rod anchor in laminated 15/30PCF Sawbones foam in standard holes.

Hybrid Prototype 3, and the 1.91 mm and 2.7 mm SMP rod anchors, were tested for pull-out strength as described in Example 7 but in this case using a 15PCF “Sawbones” solid rigid polyurethane foam laminated with a 2 mm thick layer of 30PCF foam. This model represented normal quality cancellous bone with a denser cortical bone layer. In this case the hole size was 2.6 mm. The BIORAPTOR™ control was tested dry and wet. All the SMP-containing anchors were tested wet before or after incubation at 37° C. to activate the SMP.

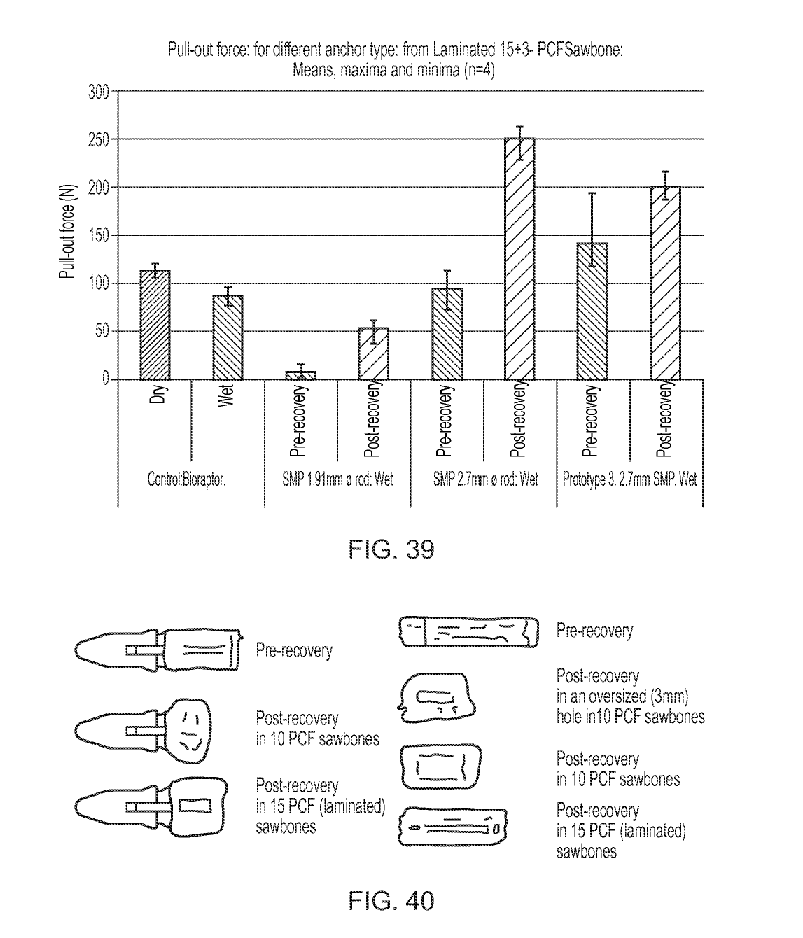

The results of the pull-out testing are shown in Table 4 and FIG. 39.

TABLE 4
Pull-out testing in laminated 15/30PCF “Sawbones” foam

| | Test | Pull out force ( [symbol] ) | | | | | | | |
|---|---|---|---|---|---|---|---|---|---|
| Anchor used | Conditions | 1 | 2 | 3 | 4 | Mean | Max | Min | SD |
| Control: Bioraptor. | Dry | [symbol] | [symbol] | [symbol] | 111.4 | 112.7 | 118.3 | 105.3 | [symbol] |
| | Wet | [symbol] | [symbol] | 76.2 | 88.6 | [symbol] | 94.0 | 76.2 | [symbol] |
| SMP 1.91 mm [symbol] rods. Wet | Pre-recovery | 7.6 | 12.9 | 4.9 | [symbol] | 7.1 | [symbol] | 3.0 | 4.31 |
| | Post-recovery | [symbol] | [symbol] | [symbol] | [symbol] | [symbol] | [symbol] | [symbol] | 10.44 |
| SMP 2.7 mm [symbol] rods. Wet | Pre-recovery | [symbol] | 111.7 | [symbol] | 72.0 | 93.2 | 111.7 | 72.0 | [symbol] |
| | Post-recovery | [symbol] | [symbol] | 251.1 | [symbol] | [symbol] | [symbol] | 228.7 | [symbol] |
| Prototype 3. 2.7 mm SMP. Wet | Pre-recovery | [symbol] | 121.1 | [symbol] | [symbol] | 142.5 | [symbol] | [symbol] | 36.74 |
| | Post-recovery | [symbol] | [symbol] | 204.4 | [symbol] | 199.7 | [symbol] | [symbol] | [symbol] |

[symbol]

indicates data missing or illegible when filed

As shown in Table 4 and FIG. 39, pull-out forces were much higher than in the “poor quality bone” (10PCF foam) model.

The 1.91 mm SMP rod anchor again showed its ability to have good fixation in an oversized (2.6 mm) hole.

The 2.7 mm SMP rod anchor had a 185% increase in pull-out strength compared to the control BIORAPTOR anchor post-recovery.

The 2.7 mm hybrid anchor—Prototype 3—had a 128% increase in pull-out-strength post-recovery compared to the BIORAPTOR control.

### Example 10

### Pull-Out Testing of 0.71 mm Diameter SMP Rod Anchors in 10PCF “Sawbones” Foam

The 0.71 mm SMP rod anchors were difficult to handle and test due to their small size. They were tested for pull-out strength as described in Example 7 with 10PCF “Sawbones” solid rigid polyurethane foam with 1 mm and 1.5 mm holes. The results are shown in Table 5.

TABLE 5
Pull-out testing of 0.71 mm SMP rod anchors in 10PCF “Sawbones” foam

| | Test | Pull out force ( [symbol] ) | | | | | | | |
|---|---|---|---|---|---|---|---|---|---|
| Anchor used | Conditions | 1 | 2 | 3 | 4 | Mean | Max | Min | SD |
| Control: Bioraptor | Dry | 18.0 | 18.0 | 13.7 | 12.5 | [symbol] | 18.0 | 12.5 | 2.85 |
| In a standard 2.6 mm hole | Wet | 11.6 | 13.1 | 10.4 | 15.6 | 12.7 | 15.6 | 10.4 | 2.25 |
| 0.71 mm [symbol] SMP rod. | 1.5 mm hole | 4.9 | 4.0 | 3.8 | 3.7 | 4.1 | 4.9 | 3.7 | 0.54 |
| Post recovery (wet). | 1.0 mm hole | 8.6 | 10.8 | 6.4 | [symbol] | 7.8 | 10.8 | 5.5 | 2.38 |

[symbol]

indicates data missing or illegible when filed

Although the pull-out forces are lower than the BIORAPTOR™ control the results show that the SMP device can have appreciable fixation even in oversized holes. Furthermore, while the pull-out strength in the 1.0 mm hole is approximately 60% that of the BIORAPTOR, the size of the SMP device is only around one quarter that of the control.

### Example 11

### Appearance of Devices Post-Recovery

After pull-out testing samples were photographed to record their appearance and the degree of shape-change. Examples are shown in FIG. 40.

In all cases, during shape recovery, the SMP in the devices expanded to a larger diameter than the insertion hole. This accounts for the increase in pull-out force observed compared to the control and pre-recovery devices. The SMP did not expand as far in in the 15 PCF sawbones as in the 10 PCF. This is because the 15 PCF sawbones is denser and hence stiffer than the 10 PCF Sawbones, giving more resistance to the expansion of the SMP.

### Example 12

### Knotless Suture Anchor

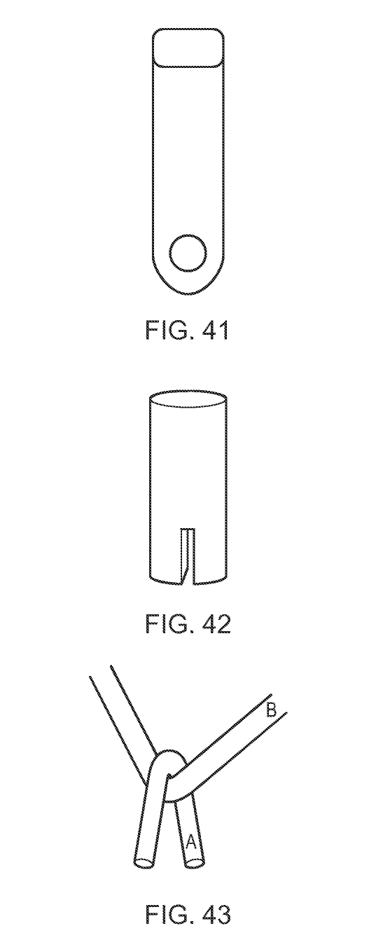

4.3 mm diameter Poly(D,L lactide-co-glycolide) 85:15 rods were prepared using a twin screw extruder. The rods were then die drawn through a 2.0 mm die at 85° C. at a rate of 30 mm min⁻¹, yielding SMP rod with a diameter of approx. 1.35 mm. The SMP rod was cut to produce approx. 10 mm lengths and pressed to produce a more rectangular cross section: The SMP rod and 0.8 mm thick metal shims were placed between metal plates and pressed with a force of 50 kN at 20° C. A 1.0 mm diameter hole was then drilled in the bottom of the device then cleaned up using a scalpel and the end of the device rounded using needle files to yield devices typically 10.20 long, 1.89 mm wide and 0.83 mm thick (FIG. 41). Two lengths of size 1 ULTRABRAID™ (Smith and Nephew) were then threaded through the hole in the device.

The device was implanted into a 1.8 mm diameter 15 mm deep hole in a 15 PCF Sawbones block so that the top of the device was 2 mm below the surface of the block.

The sutures were marked where they entered the implantation hole, red on one side, blue on the other. Holding the device in place within the hole the sutures were pulled one at a time from the blue side, to pull them through the device. Movement of the markings on the sutures demonstrating that they moved freely through the implanted device and that the tension could be adjusted. The devices were activated to lock the sutures and fix the anchor by immersing the Sawbones block in water and incubating at 37° C. for 3 days.

To simulate the use of the device in-vivo, the suture limbs on the side on which they were pulled were cut with a scalpel, leaving only the two “tensioned” limbs.

The fixation strength of the devices was tested by pulling them out of the block and measuring the force required. To do this both suture legs were pulled at the same time, at a rate of 508 mm min-1 (20 inches min-1). Three devices were tested and a mean maximum pull-out force of 45.0 N was recorded.

### Example 13

### Small Suture Anchor Example 1 (1.4 mm Diameter Hole)

4.3 mm diameter Poly(D,L lactide-co-glycolide) 85:15 rods were prepared using a twin screw extruder. The rods were then die drawn through a 2.0 mm die at 85° C. at a rate of 30 mm min⁻¹, yielding SMP rod with a diameter of approx. 1.35 mm. These rods were then cut to produce 7 mm lengths and a slot cut in the bottom (FIG. 42) into which was fitted a #2 ULTRABRAID™ suture (Smith and Nephew).

The device was inserted by placing it in a syringe needle with the point removed, holding the needle against a 1.4 mm diameter, 8 mm deep hole in 15 PCF sawbones and pushing the device in to the hole by inserting a metal rod down the syringe needle. The devices were activated by immersing the Sawbones block in water and incubating at 37° for 2 days. The fixation strength of the devices was tested by pulling them out of the block and measuring the force required. To do this both ends of the suture were pulled at the same time, at a rate of 508 mm min-1 (20 inches min-1). Four devices were tested and mean maximum pull-out force of 43.8 N was recorded.

### Example 14

1.60 mm diameter Poly(D,L lactide-co-glycolide) 85:15 rods were prepared using a twin screw extruder. The rods were then die drawn through a 0.75 mm die at 80° C. at a rate of 30 mm min⁻¹, yielding SMP rod with a diameter of approx. 0.58 mm. These rods were then cut to produce 12 mm lengths which were folded in half (FIG. 43 represented by “A”) and mounted over a USP size 1 braided multifilament suture (Trogue Ref:75221) (FIG. 43—see “B”).

The device was then mounted in a delivery tube, such that the two ends of the device were held parallel, protruding from the end with the suture gripped between them. The device was delivered into a 1 mm diameter, 8 mm deep hole in 15 PCF Sawbones, by placing the device ends into the hole, then and pushing the device in to the hole by inserting a metal rod down the delivery tube. Three devices were prepared and activated by immersing the Sawbones block in water and incubating at 37° for 3 days. The fixation strength of the devices was tested by pulling them out of the block and measuring the force required. To do this both ends of the suture were pulled at the same time, at a rate of 508 mm min-1 (20 inches min-1). A mean maximum pull-out force of 20.3 N was recorded.

### Example 15

### Use of Overmoulding to Produce Hybrid Device

An over-moulding tool for a screw with a thread was made from steel. A length of polyurethane (PU) die-drawn billet was placed in the mould and overmoulded with the same PU polymer using the Cincinnati Milacron injection moulding machine to produce an overmoulded screw. Immersion in hot water again showed shape recovery of the overmoulded screw.

A screw made in this way was cut in half and polished with diamond paste to reveal its cross-section. This was examined using scanning electron microscopy. No boundary was visible between the die-drawn SMP rod and the overmoulded polymer.

The examples show that suture anchors incorporating SMP show:
Increased fixation strength especially in poor quality bone;
Fixation strength that is largely independent of bone quality;
Fixation strength that is tolerant of overdrilling of the hole;
Small anchors, with diameter less than 2 mm e.g. 1 mm, 1.2, 1.4. 1.6 or 1.8 mm, are feasible; and
Cavities of less than 2 mm diameter can be made in the bone e.g. cavities of 1 mm, 1.2 mm, 1.4 mm, 1.6 mm or 1.8 mm.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1-42. (canceled)

43. A fixation device for use to secure itself and/or a further device in a cavity, the fixation device comprising a portion comprising a Shape Memory Polymer (SMP) material, and a further portion comprising a non-SMP material which non-SMP material is formed over at least one surface of the SMP comprising portion, by a process of overmoulding.

44. The fixation device according to claim 43, which is selected from a pin, a screw, a rod, a nail, a plate, a suture anchor, an anchor, and a wedge.

45. The fixation device according to claim 44, which is a suture anchor and is for fixing in a cavity in a bone.

46. The fixation device according to claim 43, wherein the SMP material comprises one or more of a poly(D,L-lactide) copolymers.

47. The fixation device according to claim 45, wherein the suture anchor comprises an anchor body comprising a distal portion and a proximal portion.

48. The fixation device according to claim 47, wherein the anchor body comprises a passage extending from the distal portion toward the proximal portion.

49. The fixation device according to claim 48, wherein the passage is a through passage.

50. The fixation device according to claim 47, wherein the anchor body comprises one or more circumferential ribs.

51. The fixation device according to claim 47, wherein the anchor body comprises screw threads along its length.

52. The fixation device according to claim 47, wherein the anchor body is formed integrally from a single piece of SMP material.

53. The fixation device according to claim 47, wherein the anchor body comprises SMP material and non-SMP material.

54. The fixation device according to claim 53, which comprises one or more circumferential ribs composed of the non-SMP material.

55. The fixation device according to claim 53, wherein the SMP material comprises a polymer selected from the group consisting of polymethyl methacrylate (PMMA), polyethyl methacrylate (PEMA), polyacrylate, poly-alpha-hydroxy acids, polycapropactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, polyurethanes, and copolymers or polymer blends thereof.

56. The fixation device according to claim 53, wherein the SMP material comprises one or more of a poly(D,L-lactide) copolymer.

57. The fixation device according to claim 43, wherein the SMP material comprises a poly(DL-lactide-co-glycolide) (PDLGA) co polymer.

58. The fixation device according to claim 43, wherein the SMP material further comprises a filler.

59. The fixation device according to claim 43, further wherein the SMP material comprises a bioceramic material selected from the group consisting of calcium phosphate, calcium carbonate, calcium sulphate, and combinations thereof.

60. The fixation device according to claim 43, wherein the SMP material further comprises a plasticizer, a bioactive agent and/or a pharmaceutical agent.

61. The fixation device according to claim 43, wherein the non-SMP material is resorbable.

62. The fixation device according to claim 43, wherein the non-SMP material is selected from polylactide, polyglycolide, polycaprolactone, poly(lactide-coglycolide), polydioxanone, polyurethane, a blend of one or more thereof, and copolymers thereof.

63. The fixation device according to claim 43, wherein the non-SMP material is non-resorbable.

64. The fixation device according to claim 47, wherein the non-SMP material is a nonresorbable polymer selected from the group consisting of polyetheretherketone (PEEK), a polyurethane and a polyacrylate.

65. A method of repairing a soft tissue comprising; placing a device according to claim 43 in a bone, passing a flexible member through a soft tissue located adjacent to the bone; tying the flexible member to secure the soft tissue to the body and activating the SMP material such that the device undergoes a radial expansion in at least a section of its length.

66. The method according to claim 65, comprising a first step of forming a cavity in the bone and placing the device in the cavity.

67. The method according to claim 65, wherein the soft tissue is selected from a tendon, a ligament, a muscle, cartilage, and combinations thereof.

68. A fixation device for use to secure itself and/or a further device in a cavity, the fixation device comprising a portion comprising SMP material, wherein the device is a suture anchor and further comprises one or more grooves on at least one outer surface of the suture anchor, the groove(s) sized to accommodate a suture.

69. The fixation device according to claim 68, which further comprises the one or more grooves extending down a first lateral surface of the device, along a distal end of the device, and up an opposing lateral surface of the device.

70. The fixation device according to claim 69, wherein the suture anchor comprises an anchor body comprising a distal portion and a proximal portion.

71. The fixation device according to claim 70, wherein the anchor body comprises a passage extending from the distal portion toward the proximal portion.

72. The fixation device according to claim 68, wherein the anchor body is formed integrally from a single piece of SMP material.

73. A method of repairing a soft tissue comprising placing a device according to claim 68 in a bone, passing a flexible member through a soft tissue located adjacent to the bone, tying the flexible member to secure the soft tissue to the body, and activating the SMP material such that the device undergoes a radial expansion in at least a section of its length.
